(19) United States
(12) Patent Application Publication
(10) Pub. No.: US 2024/0400559 A1
(43) Pub. Date: Dec. 5, 2024

(54) SOLID STATE EMITTERS BASED ON PYRIDINIUM SCAFFOLDS

(71) Applicant: Council of Scientific and Industrial Research, New Delhi (IN)

(72) Inventors: Beeran SENTHILKUMAR, Pune (IN); Ravindra MULE, Pune (IN); Nitin Tukaram PATIL, Bhopal (IN)

(21) Appl. No.: 18/689,213

(22) PCT Filed: Sep. 5, 2022
(86) PCT No.: PCT/IN2022/050792

§ 371 Date: Mar. 5, 2024

(30) Foreign Application Priority Data
Sep. 5, 2021 (IN) 202111040211

(51) Int. Cl. C07D 471/04; C07D 213/16; C09K 11/06

(52) U.S. Cl. CPC C07D 471/04; C07D 213/16; C09K 11/06; C09K 2211/1007; C09K 2211/1018

(57) ABSTRACT

The present invention discloses the design Solid State Luminogens based on pyridinum scaffolds. Particularly, the present invention discloses the design and development of novel N-doped ionic solid state emitters of general Formula (I) and to the process for preparation thereof. Variation in substituents as well counter ions of pyridinium scaffolds modulates physical properties of general Formula (I).

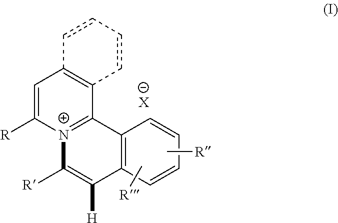

### CROSS REFERENCES TO RELATED APPLICATIONS

This application is a national-stage application under 35 U.S.C. § 371 of International Application No. PCT/IN2022/050792, filed Sep. 5, 2022, which International Application claims benefit of priority to Indian patent application Ser. No. 20/211,1040211, filed Sep. 5, 2021.

### TECHNICAL FIELD

The present disclosure relates to solid state emitters based of pyridinium derivatives. Particularly, the present disclosure relates to N-doped solid-state emitters of general Formula (I) and to the process for preparation thereof.

### BACKGROUND

Design and synthesis of luminescent organic materials is of fundamental importance because of their potential application in biology, chemistry, and optoelectronic device. In recent years, ionic solid fluorophores are gaining much attention because of their unique properties such as high thermal stabilities, phase tunabilities and water solubilities. Inspired by these features their application are in mounting a number of fields including material science and biology. As a subset, quaternary nitrogen containing aromatic heterocycles represent an important class of molecules that have interesting applications in the field of optoelectronics. light emitting-diodes, super capacitors and bio-imaging.

Jie Zhang research group in 2014, [Chem. Commun., 2014, 50, 15878] reported synthesis of pyridinium molecules which have bright white light emission by aggregation. Intramolecular charge transfer has been studied via Photophysical, single-crystal structural, and computational studies. It demonstrated that an additional low-energy emission was generated by the excitation of a new intermolecular charge-transfer (CT) band at the ground state that cooperates with the non-quenched high-energy monomer emission to produce white light, shown in the Scheme 1 below:

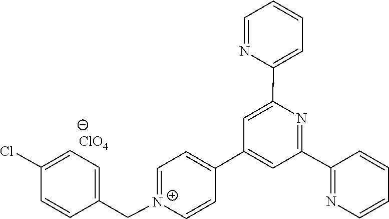

Recently Suna et al. [Chem. Commun., 2019,55, 12663-12666] reported aggregation induced emission by pyridinium-pyridinium interactions through non-covalent intermolecular interactions between Py+ subunits, later they showed a approach to achieve AIEgens by the use of non-covalent intermolecular p+-p interactions between quaternary Py+ or Im+ (imidazolium) cations and aromatic pi systems.

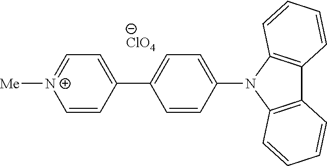

To facilitate advancement in this field the present inventors developed a novel class of redox-active N-doped ionic solid state luminogens which are structurally different from the reported pyridinium salts and wherein their photophysical properties can be tuned not only by varying substituents on the core scaffold but also with a variation of the counter anions.

### SUMMARY

It therefore remains the primary objective of this disclosure to provide a class of redox-active N-doped ionic solid state luminogens having photophysical properties that can be tuned not only by varying substituents on the core scaffold but also with a variation of the counter anions.

Yet another objective is to provide a facile process for synthesis which can generate library of redox-active N-doped ionic solid state luminogens of pyridinium scaffold.

In accordance with the above, embodiments herein provide redox-active N-doped ionic solid state luminogens of pyridinium scaffold of general formula (I);

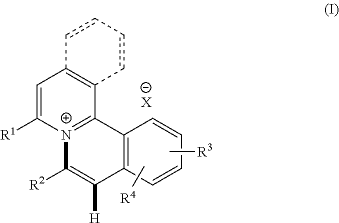

### Wherein,

R1 and R2 each independently represents hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N;
R³ and R⁴ represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N, halogen, nitrile, acetyl, hydroxyl, alkoxy, amino, nitro, carbonyl, ester or thio;
the dashed ring indicates the saturated or unsaturated six-membered cyclic ring that may be present;
X⁻ represents the counter ions selected from ⁻OTf, NTf₂⁻, SbF₆⁻, BF₄⁻, or NO₃⁻.

In another aspect a process for preparation of library redox-active N-doped ionic solid state luminogens general formula (I) comprises:
(i) preparing 2-(2-alkynylphenyl)pyridines of Formula (1); and
(ii) promoting AgX endo-selective hydroamination of 2-(2-alkynylphenyl)pyridines of Formula (1) in a solvent to obtain the desired solid state emitters of formula (I),

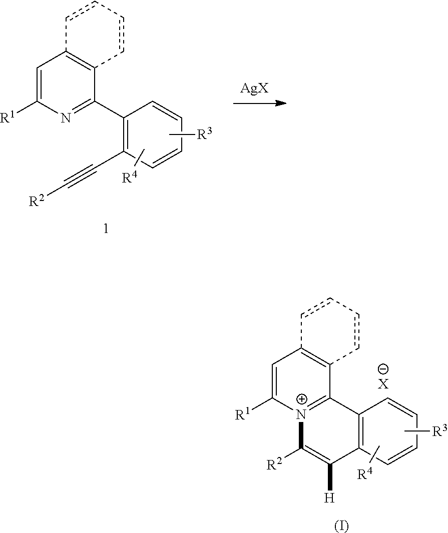

### Wherein,

R¹ and R² each independently represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N;
R³ and R⁴ represent hydrogen, (un)substituted C1-C6 alkyl, (un)substituted aryl. alkylaryl, or heteroaryl; (un)substituted C3-C8 saturated or unsaturated cycloalkyls or fused cycloalkyls optionally having heteroatoms selected from O, S or N; halogen, nitrile, acetyl, hydroxyl, alkoxy, amino, nitro, carbonyl, ester or thio;
the dashed ring indicates the saturated or unsaturated six-membered cyclic ring that may be present;
X⁻ represents the counterions selected from ⁻OTf, NTF₂⁻, SbF₆⁻, BF₄⁻, or NO₃⁻; and
AgX represent the salt selected from AgOTf, AgNTf₂, AgSbF₆, AgBF₄, or AgNO₃.

The compounds of formula (I) fluoresce in solid state and can have potential applications in bio-imaging.

### BRIEF DESCRIPTION OF THE DRAWINGS

FIG. 1 represents (a) Normalized absorption, and (b) emission spectra of the luminogens in the solid state, (c) Digital fluorescence images of the powder samples of the luminogens herein, collected upon excitation at 365 nm.

FIG. 2 represents (a), (c) Fluorescence spectra of 2f and 2u in DCM/hexane solvent mixture with increasing ƒₕₑₓₐₙₑ (0-99%) in DCM. (b), (d) PL intensity change at a certain wavelength with increasing ƒₕₑₓₐₙₑ (0-99%) in DCM for 2f and 2u respectively; insets of (b) and (d) present the fluorescence images of respective solution with the variation of ƒₕₑₓₐₙₑ under UV light (λₑₓ=365 nm) exposure.

FIG. 3 represents (a) Absorption, and (b) fluorescence spectra of compounds 3a-d containing respective counter-ion mentioned in solid state, (c) Fluorescence spectra of compound 3a in DCM/hexane solvent mixture with increasing fₕₑₓₐₙₑ (0-99%) in DCM. (d) Fluorescence intensity change at a certain wavelength with properties in both solution and the solid state, (e) Digital fluorescence images of the powder samples of luminogens herein, containing different counter-ions collected upon excitation at 365 nm.

FIG. 4 represents cytotoxicity assay of 2m and 2u in different concentrations.

FIG. 5 represents CLSM images of BHK-21 cells stained (incubation time: 15 min) with (a) 2 μM 2m. (b) 0.3 μM of Mitotracker Red, (c) merge image of (a) and (b), (d) scatter plot showing Pearson's correlation coefficient of 0.94±0.04, (f) 0.2 μM 2u, (g) 0.3 μM of Mitotracker Red, (h) merge image of (f) and (g), (i) scatter plot showing Pearson's correlation coefficient of 0.95±0.02. (Scale bar: 10 μm).

FIG. 6 represents the AgX endo-selective hydroamination of 2-(2-alkynylphenyl)pyridines of Formula (1) in a solvent to obtain the desired solid state emitters of Formula (I)

### DETAILED DESCRIPTION

The present invention will now be will now be explained in detail with reference to its various preferred as well as optional embodiment, which, however should not be construed to limit the scope of the invention.

Embodiments herein relate to N-doped ionic solid state luminogens of pyridinium scaffold, wherein, the photophysical properties can be tuned not only by varying the substituents on the core scaffold but also with a variation of the orientation of the counter anions and the corresponding non-covalent contacts present between the contact ion-pairs.

In an embodiment, redox-active N-doped ionic solid state luminogens of pyridinium scaffold have general formula (I);

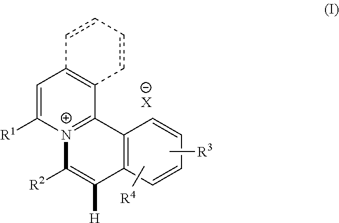

wherein,
R¹ and R² each independently represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N;
R³ and R⁴ represent hydrogen, (un)substituted C1-C6 alkyl, (un)substituted C1-C6 alkoxy, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N, halogen, nitrile, acetyl, hydroxyl, alkoxy, amino, nitro, carbonyl, ester or thio;
the dashed ring indicates the saturated or unsaturated six-membered cyclic ring that may be present;
X represent the counter-ions selected from ⁻OTf, NTF₂⁻, SbF₆⁻, BF₄⁻, or NO₃⁻.

In another embodiment, the redox-active N-doped ionic solid state luminogens of pyridinium compound have formula (Ia),

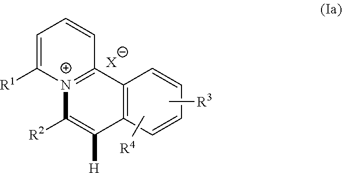

wherein,
R¹ and R² each independently represents hydrogen, (un)substituted C1-C6 alkyl, (un)substituted aryl. (un)substituted alkylaryl (un)substituted heteroaryl. (un)substituted C3-C8 saturated or unsaturated cycloalkyls or fused cycloalkyls optionally having heteroatoms selected from O, S or N;
R³ and R⁴ represent hydrogen, (un)substituted C1-C6 alkyl, (un)substituted aryl. (un)substituted alkylaryl. (un)substituted heteroaryl, (un)substituted C3-C8 saturated or unsaturated cycloalkyls or fused cycloalkyls optionally having heteroatoms selected from O, S or N, halogen, nitrile, acetyl, hydroxyl, alkoxy, amino, nitro, carbonyl, ester or thio; and
X represents the counter ions selected from ⁻OTf, NTF₂⁻, SbF₆⁻, BF₄⁻, or NO₃⁻.

In an embodiment, R¹ is (un)substituted C1-C6 alkyl, (un)substituted aryl or (un)substituted heteroaryl.

In another embodiment, R² is (un)substituted C1-C6 alkyl, (un)substituted aryl, or (un)substituted cycloalkyl.

In another embodiment, R³ is hydrogen, (un)substituted C1-C6 alkoxy, (un)substituted C1-C6 alkyl, halogen, or (un)substituted aryl.

In another embodiment, R⁴ is hydrogen, (un)substituted C1-C6 alkoxy, (un)substituted C1-C6 alkyl, halogen, or (un)substituted aryl.

In another embodiment, R¹ is methyl, phenyl, tolyl, biphenyl, cyanophenyl, methoxyphenyl, dimethoxyphenyl, acetylphenyl, (9H-carbazol-9-yl)phenyl, anthracenyl. phenanthrenyl, and benzo[b]thiophenyl.

In another embodiment, R² is phenyl, tolyl, methoxyphenyl, dimethoxyphenyl, chlorophenyl, 2-chloro-5-methoxyphenyl, napthenyl, and cyclopropyl.

In another embodiment, R³ is hydrogen, methoxy, methyl, chloro, naphthyl, phenyl, and tert-butyl.

In another embodiment, R⁴ is hydrogen, methoxy, methyl, chloro, naphthyl, phenyl, and tert-butyl.

In an aspect, the compounds of Formula (I) comprises:
i. 4,6-diphenylpyrido[2,1-a]isoquinolin-5-ium trifluoromethansulfonate (2a);
ii. 6-phenyl-4-(p-tolyl)pyrido[2,1-a]isoquinolin-5-iumtrifluoromethane sulfonate (2b);
iii. 4-([1.1′-biphenyl]-4-yl)-6-phenylpyrido[2,1-a]isoquinolin-5-ium trifluoro methane sulfonate (2c);
iv. 4-(4-cyanophenyl)-6-phenylpyrido[2,1-a]isoquinolin-5-ium trifluoro methane sulfonate (2d);
V. 4-(4-acetylphenyl)-6-phenylpyrido[2,1-a]isoquinolin-5-ium trifluoro methane sulfonate (2e);
vi. 4-(4-methoxyphenyl)-6-phenylpyrido[2,1-a]isoquinolin-5-ium trifluoro methanesulfonate (2f);
vii. 4-(4-(9H-carbazol-9-yl)phenyl)-6-phenylpyrido[2,1-a]isoquinolin-5-ium trifluoro methanesulfonate (2g);
viii. 4-(3,5-dimethoxyphenyl)-6-phenylpyrido[2,1-a]isoquinolin-5-ium trifluoro methanesulfonate (2h);
ix. 4-(anthracen-9-yl)-6-phenylpyrido[2,1-a]isoquinolin-5-ium trifluoro methanesulfonate (2i);
X. 4-(phenanthren-9-yl)-6-phenylpyrido[2,1-a]isoquinolin-5-ium trifluoro methanesulfonate (2j);
xi. 4-(benzo[b]thiophen-2-yl)-6-phenylpyrido[2,1-a]isoquinolin-5-ium trifluoro methanesulfonate (2k);
xii. 4-(9H-carbazol-9-yl)-6-phenylpyrido[2,1-a]isoquinolin-5-ium trifluoro methanesulfonate (2l);
xiii. 4-(4-methoxyphenyl)-10-methyl-6-phenylpyrido[2,1-a]isoquinolin-5-ium trifluoromethanesulfonate (2m);
xiv. 10-(tert-butyl)-4-(4-methoxyphenyl)-6-phenylpyrido[2,1-a]isoquinolin-5-ium trifluoromethanesulfonate (2n);
XV. 4-(4-methoxyphenyl)-6,10-diphenylpyrido[2,1-a]isoquinolin-5-ium trifluoromethanesulfonate (2o);
xvi 4-(4-methoxyphenyl)-10-(naphthalen-1-yl)-6-phenylpyrido[2,1-a] isoquinolin-5-ium trifluoromethanesulfonate (2p);
xvii. 10-chloro-4-(4-methoxyphenyl)-6-phenylpyrido[2,1-a]isoquinolin-5-ium trifluoromethanesulfonate (2q);
xviii. 4-(4-methoxyphenyl)-9-methyl-6-phenylpyrido[2,1-a]isoquinolin-5-ium trifluoromethanesulfonate (2r);
xix. 9-chloro-4-(4-methoxyphenyl)-6-phenylpyrido[2,1-a]isoquinolin-5-ium trifluoromethanesulfonate (2s);
XX. 4-(4-methoxyphenyl)-8,10-dimethyl-6-phenylpyrido[2,1-a]isoquinolin-5-ium trifluoromethanesulfonate (2t);
xxi. 8,10-dimethoxy-4-(4-methoxyphenyl)-6-phenylpyrido[2,1-a]isoquinolin-5-ium trifluoromethanesulfonate (2u);
xxii. 4-(4-methoxyphenyl)-6-(p-tolyl)pyrido[2,1-a]isoquinolin-5-ium trifluoromethanesulfonate (2v);
xxiii. 6-(2-methoxyphenyl)-4-(4-methoxyphenyl)pyrido[2,1-a]isoquinolin-5-ium trifluoromethanesulfonate (2w);
xxiv. 6-(4-chlorophenyl)-4-(4-methoxyphenyl)pyrido[2,1-a]isoquinolin-5-ium trifluoromethanesulfonate (2x);
XXV. 6-(3,5-dimethoxyphenyl)-4-(4-methoxyphenyl)pyrido[2,1-a]isoquinolin-5-ium trifluoromethanesulfonate (2y);
xxvi. 6-(2-chloro-5-methoxyphenyl)-4-(4-methoxyphenyl)pyrido[2,1-a]isoquinolin-5-ium trifluoromethanesulfonate (2z);
xxvii. 4-(4-methoxyphenyl)-6-(naphthalen-1-yl)pyrido[2,1-a]isoquinolin-5-ium trifluoromethanesulfonate (2aa);
xxviii. 8-(4-methoxyphenyl)-6-phenylisoquinolino[3,2-a]isoquinolin-7-ium trifluoromethanesulfonate (2ab);
xxix. 4-methyl-6-phenylpyrido[2,1-a]isoquinolin-5-ium (2ac);
XXX. 6-cyclopropyl-4-(4-methoxyphenyl)pyrido[2,1-a]isoquinolin-5-ium trifluoromethanesulfonate (2ad);
xxxi. 4-(4-methoxyphenyl)-6-phenylpyrido[2,1-a]isoquinolin-5-ium bis((trifluoromethyl)sulfonyl)amide (3a);
xxxii. 4-(4-methoxyphenyl)-6-phenylpyrido[2,1-a]isoquinolin-5-ium hexafluorostibate (3b);
xxxiii. 4-(4-methoxyphenyl)-6-phenylpyrido[2,1-a]isoquinolin-5-ium tetrafluoroborate (3c);
xxxiv. 4-(4-methoxyphenyl)-6-phenylpyrido[2,1-a]isoquinolin-5-ium nitrate (3d).

The term, “C1-C6 alkyl”, as used herein, refers to the radical of saturated aliphatic groups, including straight or branched-chain alkyl groups having six or fewer carbon atoms in its backbone, for instance, C1-C6 alkyl for straight chain and C3-C6 for branched chain. As used herein, C1-C6 alkyl refers to an alkyl group having from 1 to 6 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “halogen” as used herein refers to chlorine, fluorine, and bromine or iodine atom.

The term, “(C1-C6) alkoxy” refers to a (C1-C6)alkyl having an oxygen radical attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆) alkoxy substituted with one or more groups, particularly one to four groups independently selected from the groups indicated above as the substituents for the alkyl group.

The term “C3-C8 saturated or unsaturated cycloalkyls” refers to a saturated or unsaturated cyclic hydrocarbon radical including 1, 2 or 3 rings and including a total of 3-8 carbon atoms forming the rings. Examples include, but are not limited to, cyclopropyl, cyclobutyl, cyclopentyl, cyclohexyl, adamantyl, norbornyl, bicyclo[2.1.0]pentane, cyclopentenyl and cyclohexenyl. Unless stated otherwise, C3-C8 saturated or unsaturated cycloalkyls can be unsubstituted or substituted with one or more substituents, for example, substituents independently selected from the group consisting of oxo, halogen, (C₁₋₆) alkyl, hydroxy, cyano, nitro and amine. Cycloalkyl group comprises a saturated cycloalkyl ring system which does not contain any double bond within the ring or a partially unsaturated cycloalkyl ring system which may contain one or more double bonds within the ring system that is stable, and do not form an aromatic ring system.

The term “aryl” as used herein refers to monocyclic, bicyclic or tricyclic hydrocarbon groups having 6 to 14 ring carbon atoms, wherein at least one carbocyclic ring is having a x electron system. Examples of aryl ring systems include, but are not limited to, phenyl, naphthy, anthracenyl and phenanthrenyl. Unless indicated otherwise, aryl group can be unsubstituted or substituted with one or more substituents, for example 1-4 substituents independently selected from the group consisting of halogen, (C1-C6) alkyl, (C1-C6)alkoxy, acetyl, 9H-carbazol-9-yl, hydroxy, phenyl, cyano, nitro,—COOH and amino.

As used herein, the term “heteroaryl” refers to a 5-14 membered aromatic monocyclic bicyclic, or tricyclic ring system containing one to four heteroatoms independently selected from: nitrogen, sulphur and oxygen. Representative examples of heteroaryls include, but are not limited to, pyrrole, pyrazole, imidazole, pyrazine, furan, thiophene, oxazole, thiazole, benzimidazole, benzoxazole, benzothiazole, benzothiophene, benzofuran, indole, indazole, isoindole, isoquinoline, isooxazole, triazine, purine, pyridine, quinoline, oxadiazole, thiene, pyridazine, pyrimidine, isothiazole, quinoxaline (benzopyrine), tetrazole and carbazole. The nitrogen or sulfur atom of the heterocyclyl can be optionally oxidized to the corresponding N-oxide, S-oxide or S, S-dioxide. The heteroaryl group can be unsubstituted or substituted with one or more groups independently selected from the group consisting of halogen, hydroxy. oxo, cyano, (C₁-C₈)-alkyl, halo(C₁-C₆)-alkyl, (C₁-C₆)-alkoxy, or halo(C₁-C₆)-alkoxy. The substituents can be present on either the ring carbon or the ring nitrogen atom(s). The substituents can be present at one or more positions provided that a stable molecule results.

In yet another embodiment, a process for synthesis of library of redox-active N-doped ionic solid state luminogens of pyridinium scaffold of general formula (I) comprises;
i. preparing 2-(2-alkynylphenyl)pyridines of Formula (1); and
ii. promoting AgX endo-selective hydroamination of 2-(2-alkynylphenyl)pyridines of Formula (1) in a solvent to obtain the desired solid state emitters of formula (I) heating the compound of formula (1) with lequiv. of AgX in moist DCE at about 80° C. for about 12 hours to obtain the desired AIEgens of formula (I),

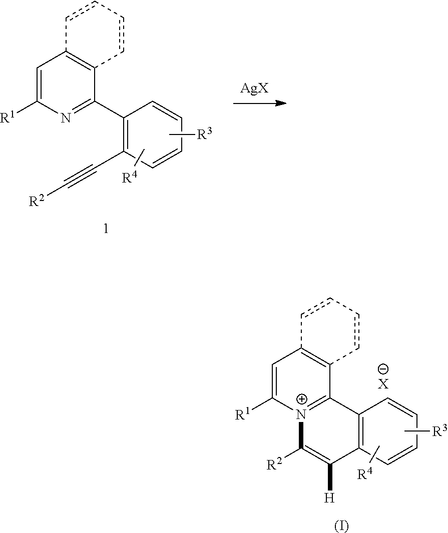

wherein,
R¹ and R² each independently represent hydrogen, (un)substituted C1-C6 alkyl, (un)substituted aryl, (un)substituted alkylaryl, (un)substituted heteroaryl, (un)substituted C3-C8saturated or unsaturated cycloalkyls or fused cycloalkyls optionally having heteroatoms selected from O, S or N,
R³ and R⁴ represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N, halogen, nitrile, acetyl, hydroxyl, alkoxy, amino, nitro, carbonyl, ester or thio;
the dashed ring indicates the saturated or unsaturated six-membered cyclic ring that may be present;
X⁻ represent the counterions selected from ⁻OTf, NTf₂⁻, SbF₆⁻, BF₄⁻, or NO₃⁻; and
AgX represent the salt selected from AgOTf, AgNTf₂, AgSbF₆, AgBF₄, or AgNO₃.

In a preferred embodiment, the promoting process step b) is done by heating the compound of formula (1) with 1 equiv of AgX in solvent at a temperature of 80° C. for about 12 hours to obtain the desired AIEgen compounds of formula (I).

In another preferred embodiment, the solvent used in promoting step b) is DCE or chloroform.

In yet another embodiment, a process for preparation of compound of formula (I) comprises the step of heating the compound of formula (1) with 1 equiv. of AgX in moist DCE at about 80° C. for about 12 hours to obtain the compound of formula (I),

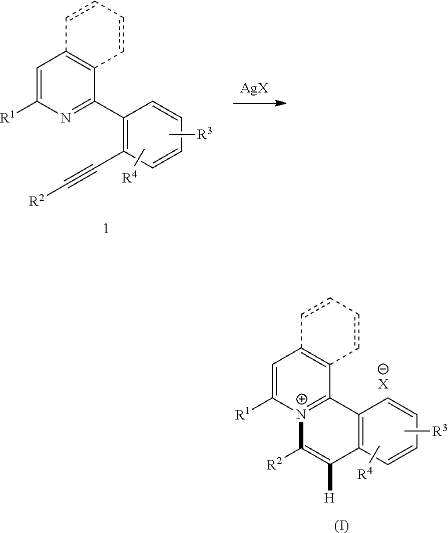

wherein R¹, R², R³, R⁴, and X are as defined earlier.

In another embodiments, the 2-aryl 6-(2(alkynyl)-aryl) pyridines of formula (1) is prepared by a process known in the art or by modifying the known process to obtain the desired compounds in good yield and purity. Accordingly, the 2-aryl 6-(2(alkynyl)-aryl) pyridines of formula 1a-lab, 1f, 1h-1k, 1m, and 1q-1aa which are reported in the literature are prepared according to the literature known procedures. The 2-aryl 6-(2(alkynyl)-aryl) pyridines of formula 1c-le, 1g, 1I, 1n-1p and 1ab are prepared from slight modifications in literature known procedures.

The compounds of formula 1c-le, 1g, 1I, 1n-1p and 1ab are prepared by the process comprising;
(i) Sonogashira cross-coupling of 2-bromo-1-iodobenzene of formula (4), PdCl₂(PPh₃)₂, Cu(I) iodide in base with aryl acetylene of formula (5) to yield substituted 2-alkynyl-bromobenzenes of formula (6);

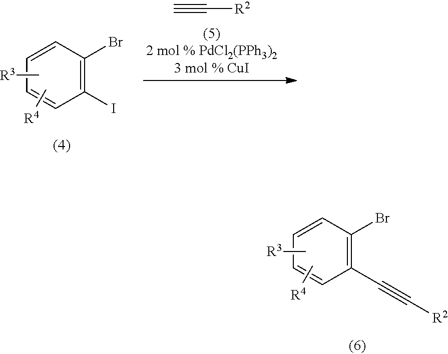

(ii) Lithiation, borylation of compound (6) to obtain 2-alkynylphenylboronic acids of formula (7);

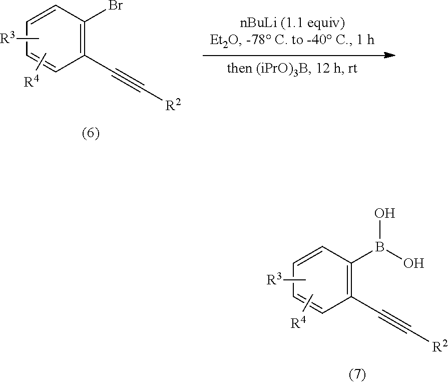

(iii) Suzuki coupling reaction of compound (8) with Di-Bromopyridine to obtain 2-(aryl)-monobromopyridines of formula (9); and

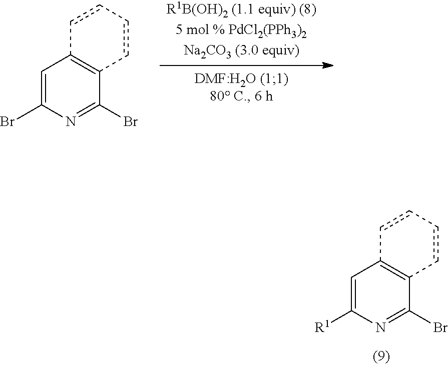

(iv) Suzuki cross-coupling reaction of compound (9) aryl boronic acid in presence of PdCl₂(PPh₃)₂, base and aq.solvent in 1:1 ratio to give the desired compounds.

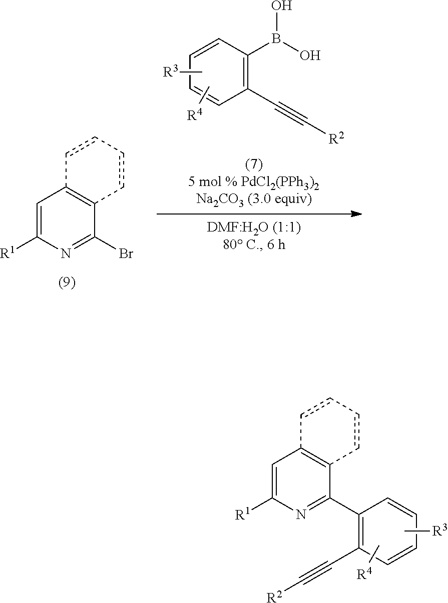

wherein,
R¹ and R² each independently represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N;
R³ and R⁴ represent hydrogen, (un)substituted C1-C6 alkyl, (un)substituted aryl, (un)substituted alkylaryl, (un)substituted heteroaryl, (un)substituted C3-C8 saturated or unsaturated cycloalkyls or fused cycloalkyls optionally having heteroatoms selected from O, S or N, halogen, nitrile, acetyl, hydroxyl, alkoxy, amino, nitro, carbonyl, ester or thio; and
the dashed ring indicates the saturated or unsaturated six-membered cyclic ring that may be present.

In another embodiment, the compounds of general formula (I) in powder form showed a bright and intense luminescence. The compounds find potential applications in bio-imaging.

### EXPERIMENTAL

### Example 1

### Synthesis of Starting Materials: Referring to the Scheme for Synthesis of Compounds 1c-1e, 1g, 1l, 1n-1p and 1ab Provided in Detailed Description

1.1: General Procedure for Preparation of Substituted 2-alkynyl-bromobenzenes

The bromo-alkynes S₁-S₈ and S₁₂-S₁₆ (See below) were reported in the literature and prepared according to the literature known procedure. The other bromo-alkynes S₁₀-S₁₂ were prepared from the literature known procedure by slightly modified procedure.

Representative procedure for Sonogashira cross-coupling: A suspension of 2-bromo-4-(tert-butyl)-1-iodobenzene (2 gm, 5.89 mmol, 1.0 equiv.), PdCl₂(PPh₃)₂ (83 mg, 0.11 mmol, 2 mol %), Cu(I) iodide (33.5 mg, 0.17 mmol, 3 mol %) in 20 mL of triethylamine was degassed three times. After 10 min a solution of phenylacetylene (0.71 mL, 6.48 mmol, 1.1 equiv) in triethylamine (3 mL) was added drop-wise over 5 min via syringe and the reaction mixture was left to stir for 12 h. After the total consumption of the 2-bromo-4-(tert-butyl)-1-iodobenzene, as monitored by TLC, the reaction mixture was filtered through celite and extracted with ethyl acetate (3×10 mL). The organic layer was washed with a saturated solution of NH₄Cl (2×10 mL), water (2×10 mL), dried over Na₂SO₄ and the solvent was removed under vacuo. The reaction mixture was purified by flash chromatography on silica gel, (eluent: petroleum ether) to give the 2-bromo-4-(tert-butyl)-1-(phenylethynyl)benzene (S₁₁) in 88% yield. Note: The Aryl bromo alkyne S₁₀ was accompanied with slight impurity and hence used directly for next reaction without attempting further purification.

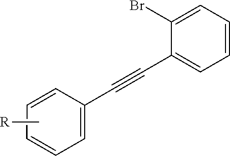

S₁, R = H, 90%
S₂, R = 4-Me, 85%
S₃, R = 2-OMe, 90%
S₄, R = 4-Cl, 68%
S₅, R = 3,5 di-OMe, 80%
S₆, R = 2-Cl, 5-OMe, 75%

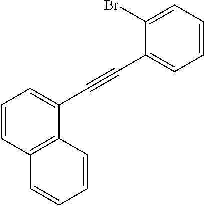 [SMILES: Brc1ccccc1C#Cc1cccc2ccccc12]

S₇, 84%

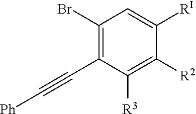

S₈, R¹ = Me, R² = R³ = H, 82%
S₉, R¹ = tBu, R² = R³ = H, 82%
S₁₀, R¹ = Ph, R² = R³ = H, 85%
S₁₁, R¹ = 1-Nap, R² = R³ = H, 88%
S₁₂, R¹ = Cl, R² = R³ = H, 78%
S₁₃, R² = Me, R¹ = R³ = H, 73%
S₁₄, R² = Cl, R¹ = R³ = H, 75%
S₁₅, R¹ = R³ = Me, R² = H, 65%
S₁₆, R¹ = R³ = OMe, R² = H, 70%

3-bromo-4-(phenylethynyl)-1,l′-biphenyl (S₁₀)

Off white solid, 1.57 gm, 85% yield; mp=74-76° C.; Rƒ=0.50 (Hexane/EtOAc=98/02); ¹H NMR (700 MHZ, CDCl₃) δ=7.90 (s, 1 H), 7.64 (d, J=7.3 Hz, 3 H), 7.60 (d, J=7.5 Hz, 2 H), 7.55 (d, J=8.0 Hz, 1 H), 7.49 (t, J=7.3 Hz, 2 H), 7.44-7.39 (m, 4 H); ¹³C NMR (175 MHz, CDCl₃) δ=142.4, 138.9, 133.4, 131.7, 130.8, 128.9, 128.6, 128.4, 128.1, 127.0, 126.0, 125.7, 124.0, 122.9, 94.4, 88.0; HRMS (ESI) calcd for C₂₀H₁₃Br (M+Na)⁺ 355.0093, found 355.0075.

1-(3-bromo-4-(phenylethynyl)phenyl)naphthalene (S₁₁)

Off white solid, 1.64 gm, 88% yield; mp=75-77° C.; Rƒ=0.60 (Hexane/EtOAc=95/05); ¹H NMR (500 MHz, CDCl₃) δ=7.99-7.91 (m, 3 H), 7.87-7.83 (m, 1H), 7.76-7.67 (m, 3 H), 7.60-7.50 (m, 3 H), 7.50-7.41 (m, 5 H); ¹³C NMR (125 MHZ, CDCl₃) δ=142.2, 137.9, 133.7, 132.8, 131.7, 131.1, 128.8, 128.6, 128.4, 128.4, 126.9, 126.4, 126.0, 125.5, 125.4, 125.3, 124.2, 122.9, 94.3, 88.0; HRMS (ESI) calcd for C₂₄H₁₅Br (M+H)⁺ 383.0412, found 383.0415.

1.2: General Procedure for Preparation of 2-alkynylphenylboronic Acids

The boronic acid S₁₇-S₂₄ and S₂₈-S₃₂ were reported in the literature and prepared according to the literature known procedure.¹ The other boronic acids S₂₅-S₂₇ were prepared from literature known procedure with slight modifications.

### Representative Procedure for Lithiation/Borylation:

In a two-necked round bottom flask, 1.6 M solution of ⁿBuLi in n hexanes (4.5 mL, 7.18 mmol, 1.5 equiv) was added drop-wise to a solution of 2-bromo-4-(tert-butyl)-1-(phenylethynyl)benzene (1.5 g, 4.78 mmol, 1.0 equiv) in 45 mL of diethyl ether under nitrogen atmosphere at −78° C. The mixture was stirred at −78° C. for 1 h and then at −40° C. for 1 h then cool back to −78° C. and B(O¹Pr)₃ (1.65 g, 8.78 mmol, 1.5 equiv) was added drop-wise. The mixture was allowed to warm up gradually to room temperature, while maintaining vigorous stirring for 16 h. Then, the reaction was quenched with 40 mL of 1N HCl for 30 minutes and extracted with ethyl acetate (3×20 mL). The combined organic solution was dried over Na₂SO₄ and the solvent was removed under vacuo. The product was purified by flash chromatography on silica gel, (eluent: petroleum ether/ethyl acetate) followed by recrystallization from petroleum ether to give the product (5-(tert-butyl)-2-(phenylethynyl)phenyl)boronic acid (S₂₅) in 80% yield. Note: The boronic acid S₂₅-S₂₇ were accompanied with slight impurities and hence used directly for next reaction without attempting further purification.

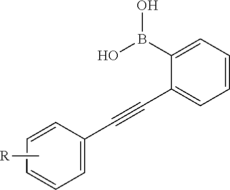

S₁₇, R = H, 74%
S₁₈, R = 4-Me, 74%
S₁₉, R = 2-OMe, 72%
S₂₀, R = 4-Cl, 68%
S₂₁, R = 3,5 di-OMe, 80%
S₂₂, R = 2-Cl, 5-OMe, 77%

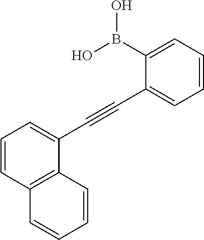 [SMILES: OB(O)c1ccccc1C#Cc1cccc2ccccc12]

S₂₃, 74%

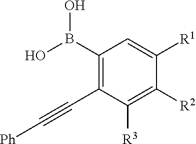

S₂₄, R¹ = Me, R² = R³ = H, 70%
S₂₅, R¹ = tBu, R² = R³ = H, 72%
S₂₆, R¹= Ph, R² = R³ = H, 70%
S₂₇, R¹ = 1-Nap, R² = R³ = H, 75%
S₂₈, R¹ = Cl, R² = R³ = H, 73%
S₂₉, R² = Me, R¹ = R³ = H, 72%
S₃₀, R² = Cl, R¹ = R³ = H, 75%
S₃₁, R¹ = R³ = Me, R² = H, 75%
S₃₂, R¹ = R³ = OMe, R² = H, 69%

1.3: Synthesis of 2-(aryl)-6-(2(alkynyl)-phenyl)pyridines

### Representative Procedure for Suzuki Cross-Coupling Reaction:

In a sealed tube 2,6-di-Bromopyridine (200 mg, 0.84 mmol, 1.0 equiv) and 4-Acetylphenyl boronic acid (139 mg, 1.0 equiv) in DMF/H₂O (1:1, 4 mL) was degassed with nitrogen for 5 min. Next, Na₂CO₃ (265 mg, 3 equiv) and PdCl₂(PPh₃)₂ (30 mg, 5 mol %) were added under nitrogen atmosphere. The reaction mixture was stirred at 80° C. for 4 h. After complete consumption of starting material, as monitored by TLC, the resulting mixture was allowed to bring to room temperature. The reaction mixture was diluted with NaHCO₃ (5 mL), and then the product was extracted with ethyl acetate (3×5 mL). The combined organic layer was dried over Na₂SO₄ and the organic solvent was removed under vacuo. The crude product was purified on a silica gel column using petroleum ether/ethyl acetate as eluent to afford 1-(4-(6-bromopyridin-2-yl)phenyl)ethan-1-one (S₃₄) in 70% yield.

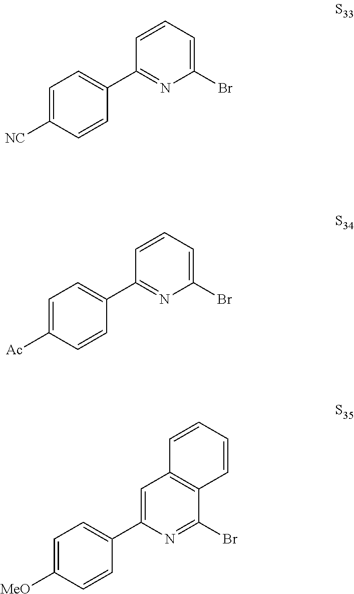 [SMILES: N#Cc1ccc(-c2cccc(Br)n2)cc1 ; CC(=O)c1ccc(-c2cccc(Br)n2)cc1 ; COc1ccc(-c2cc3ccccc3c(Br)n2)cc1]

4-(6-bromopyridin-2-yl)benzonitrile (S₃₃)

white solid, 131 mg, 65% yield; mp=95-97° C.; Rƒ=0.30 (Hexane/EtOAc=95/05); ¹H NMR (400 MHZ, CDCl₃) δ=8.12 (d, J=8.3 Hz, 2 H), 7.79-7.72 (m, 3 H), 7.70-7.64 (m, 1 H), 7.51 (d, J=7.7 Hz, 1 H); ¹³C NMR (100 MHZ, CDCl₃) δ=156.2, 142.5, 141.6, 139.3, 132.6, 127.7, 127.5, 119.5, 118.5, 113.1; HRMS (ESI) calcd for C₁₂H₇BrN₂ (M+H)⁺ 258.9865, found 258.9855.

1-(4-(6-bromopyridin-2-yl)phenyl)ethan-1-one (S₃₄)

White solid, 163 mg, 70% yield; mp=98-100° C.; Rƒ=0.50 (EtOAc/Hexane =90/10); ¹H NMR (700 MHZ, CDCl₃) δ=8.11-8.07 (m, J=8.2 Hz, 2 H), 8.06-8.02 (m, J=8.4 Hz, 2 H), 7.75 (d, J=7.5 Hz, 1 H), 7.64 (t, J=7.7 Hz, 1 H), 7.47 (d, J=7.7 Hz, 1 H), 2.65 (s, 3 H); ¹³C NMR (175 MHz, CDCl₃) δ=197.7, 157.1, 142.3, 141.6, 139.1, 137.6, 128.8, 127.2, 127.1, 119.5, 26.8; HRMS (ESI) calcd for C₁₃H₁₀BrON (M+Na)⁺ 297.9838, found 297.9858.

1-bromo-3-(4-methoxyphenyl)isoquinoline (S₃₅)

White solid, 164 mg, 75% yield; mp=104-106° C.; Rƒ=0.30 (EtOAc/Hexane =95/05); ¹H NMR (400 MHZ, CDCl₃) δ=8.17-8.09 (m, 1 H), 7.84 (s, 1 H), 7.79 (d, J=8.2 Hz, 1 H), 7.73-7.63 (m, 3 H), 7.54 (ddd, J=1.2, 7.0, 8.4 Hz, 1 H), 7.11-7.01 (m, 2 H), 3.91 (s, 3 H); ¹³C NMR (100 MHZ, CDCl₃) δ=161.3, 160.4, 139.0, 135.0, 131.5, 130.7, 130.7, 127.9, 127.4, 126.1, 125.6, 122.4, 113.9, 55.4; HRMS (ESI) calcd for C₁₆H₁₂BrON (M+Na)⁺ 335.9994, found 336.0021.

### Example 2

### Synthesis of 2-aryl 6-(2(alkynyl)-aryl)pyridines of Formula (1)

The 2-aryl 6-(2(alkynyl)-aryl)pyridines 1a-1ab, 1f, 1h-1k, 1m, and 1q-1aa were reported in the literature and prepared according to the literature known procedures ^{1a}. The other 2-aryl 6-(2(alkynyl)-aryl) pyridines 1c-1e, 1g, 1I, 1n-1p and 1ab were prepared from slight modifications in literature known procedures.

### Representative Procedure for Suzuki Cross-Coupling Reaction:

In a sealed tube 2-bromo-6-(4-methoxyphenyl)pyridine (200 mg, 0.757 mmol, 1.0 equiv) and (5-(tert-butyl)-2-(phenylethynyl)phenyl) boronic acid (229 mg, 0.825 mmol, 1.1 equiv) in DMF/H₂O (1:1 4 mL) was degassed with nitrogen for 5 min. Next, Na₂CO₃ (260 mg, 2.47 mmol, 3 equiv) and PdCl₂(PPh₃)₂ (27 mg, 0.038 mmol, 5 mol %) were added under nitrogen atmosphere. The reaction mixture was stirred at 80° C. for 6 h. After complete consumption of starting material, as monitored by TLC, the resulting mixture was allowed to bring to room temperature. The reaction mixture was diluted with NaHCO₃ (5 mL), and then the product was extracted with ethyl acetate (3×5 mL). The combined organic layer was dried over Na₂SO₄ and the organic solvent was removed under vacuo. The crude product was purified on a silica gel column using petroleum ether/ethyl acetateas eluent to afford 2-(5-(tert-butyl)-2-(phenylethynyl)phenyl)-6-(4-methoxyphenyl)pyridine (1n) in 80% yield. Note: The aryl pyridine alkyne 1ab was accompanied with slight impurity and hence used directly for next reaction without attempting further purification.

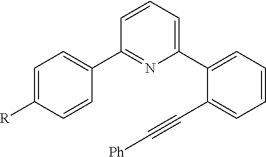

1a, R = H, 84%
1b, R = Me, 84%
1c, R = Ph, 84%
1d, R = CN, 72%
1e, R = Ac, 72%
1f, R = OMe, 90%
1g, R = 9-Carbazolyl, 72%

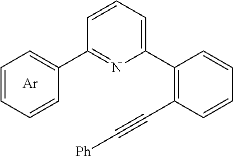

1h, Ar = 3,5 di-OMe-C₆H₃, 90%
1i, Ar = 9-Anthracenyl, 78%
1j, Ar = 9-Phenanthrenyl, 70%
1k, Ar = 2-Benzothiophenyl, 75%
1l, Ar = 9-Carbazolyl, 80%

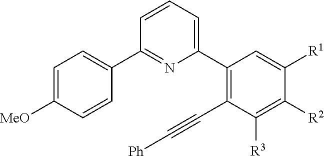

1m, R¹ = Me, R² = R³ = H, 80%
1n, R¹ = tBu, R² = R³ = H, 80%
1o, R¹ = Ph, R² = R³ = H, 80%
1p, R¹ = 1-Nap, R² = R³ = H, 80%
1q, R¹ = Cl, R² = R³ = H, 82%
1r, R² = Me, R¹ = R³ = H, 72%
1s, R² = Cl, R¹ = R³ = H, 75%
1t, R¹ = R³ = Me, R² = H, 82%
1u, R¹ = R³ = OMe, R² = H, 80%

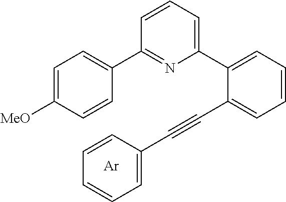

1v, Ar = 4-Me-C₆H₄, 84%
1w, Ar = 2-OMe-C₆H₄, 84%
1x, Ar = 4-Cl-C₆H₄, 78%
1y, Ar = 3,5 di-OMe-C₆H₃, 72%
1z, Ar = 2-Cl, 5-OMe-C₆H₃, 72%
1aa, Ar = 1-Nap, 84%

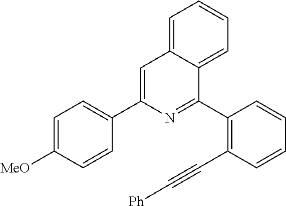 [SMILES: COc1ccc(-c2cc3ccccc3c(-c3ccccc3C#Cc3ccccc3)n2)cc1]

1ab, 76%

2-([1,1′-biphenyl]-4-yl)-6-(2-(phenylethynyl)phenyl)pyridine (1c)

Off white solid, 197 mg, 75% yield; mp=138-140° C.; Rƒ=0.30 (Hexane/EtOAc=95/05); ¹H NMR (500 MHZ, CDCl₃) δ=8.27-8.21 (m, 2 H), 8.01-7.94 (m, 2 H), 7.88 (t, J=7.8 Hz, 1 H), 7.81 (dd, J=0.8, 7.9 Hz, 1 H), 7.75-7.69 (m, 3 H), 7.69-7.65 (m, 2 H), 7.55-7.43 (m, 4 H), 7.43-7.36 (m, 3 H), 7.33-7.28 (m, 3 H); ¹³C NMR (125 MHz, CDCl₃) δ=157.4, 156.4, 142.3, 141.6, 140.7, 138.4, 136.5, 133.3, 131.4, 129.9, 128.8, 128.6, 128.3, 128.2, 128.2, 127.4, 127.4, 127.1, 123.4, 122.6, 121.4, 118.7, 92.5, 89.4; HRMS (ESI) calcd for C₃₁H₂₁N (M+H)⁺ 408.1747, found 408.1767.

4-(6-(2-(phenylethynyl)phenyl)pyridine-2-yl)benzonitrile (1d)

White solid, 165 mg, 60% yield; mp=104-106° C.; Rƒ=0.40 (Hexane/EtOAc=85/15); ¹H NMR (700 MHZ, CDCl₃) δ=8.25 (d, J=8.2 Hz, 2 H), 8.02 (d, J=8.0 Hz, 1 H), 7.94-7.87 (m, 2 H), 7.77 (d, J=7.7 Hz, 1 H), 7.74-7.70 (m, 3 H), 7.50 (t, J=7.5 Hz, 1 H), 7.45 (t, J=7.5 Hz, 1 H), 7.40-7.36 (m, 2 H), 7.35-7.28 (m, 3 H); ¹³C NMR (175 MHZ, CDCl₃) δ=157.7, 154.4, 143.4, 141.6, 136.8, 133.4, 132.5, 132.4, 131.3, 129.7, 128.6, 128.5, 128.3, 127.5, 127.4, 123.6, 123.1, 121.4, 119.4, 119.2, 118.8, 112.2, 92.6, 89.2; HRMS (ESI) calcd for C₂₆H₁₆N_{2 (M+Na)}⁺ 379.1206, found 379.1203.

1-(4-(6-(2-(phenylethynyl)phenyl)pyridine-2-yl)phenyl)ethan-1-one (1c)

White solid, 205 mg, 76% yield; mp=125-127° C.; Rƒ=0.30 (Hexane/EtOAc=90/10); ¹H NMR (700 MHz, CDCl₃) 67 =8.28-8.23 (m, J=8.2 Hz, 2 H), 8.07-8.03 (m, J=8.2 Hz, 2 H), 8.01 (d, J=7.7 Hz, 1 H), 7.93 (d, J=7.5 Hz, 1 H), 7.90 (dt, J=2.3, 7.8 Hz, 1 H), 7.81 (d, J=7.7 Hz, 1 H), 7.72 (d, J=7.5 Hz, 1 H), 7.51 (t, J=7.5 Hz, 1 H), 7.44 (t, J=7.5 Hz, 1 H), 7.41-7.36 (m, 2 H), 7.34-7.28 (m, 3 H), 2.65 (s, 3 H); 13C NMR (175 MHZ, CDCl₃) δ=197.9, 157.7, 155.5, 143.7, 142.0, 137.0, 136.6, 133.3, 131.4, 129.9, 128.7, 128.7, 128.4, 128.3, 128.3, 127.1, 123.4, 123.3, 121.4, 119.3, 92.6, 89.2, 26.7; HRMS (ESI) calcd for C₂₇H₁₉ON (M+Na)⁺ 396.1359, found 396.1359.

9-(4-(6-(2-(phenylethynyl)phenyl)pyridin-2-yl)phenyl)-9H-carbazole (1g)

White solid, 159 mg, 64% yield; mp=151-153° C.; Rƒ=0.30 (Hexane/EtOAc=85/15); ¹H NMR (400 MHZ, CDCl₃) δ=8.44-8.36 (m, 2 H), 8.22-8.15 (m, J=7.8 Hz, 2 H), 8.02-7.90 (m, 3 H), 7.89-7.83 (m, 1 H), 7.75 (dd, J=1.2, 7.6 Hz, 1 H), 7.70-7.65 (m, J=8.6 Hz, 2 H), 7.55-7.49 (m, 2 H), 7.49-7.41 (m, 6 H), 7.36-7.29 (m, 5 H); 13C NMR (175 MHz, CDCl₃) δ=157.6, 155.9, 142.2, 140.7, 138.4, 138.2, 136.7, 133.4, 131.4, 129.9, 128.7, 128.5, 128.3, 128.3, 128.2, 127.0, 126.0, 123.4, 123.4, 122.7, 121.5, 120.3, 120.0, 118.8, 109.8, 92.5, 89.4; HRMS (ESI) calcd for C₃₇H₂₄N_{2 (M+H)}⁺497.2012, found 497.1990.

9-(6-(2-(phenylethynyl)phenyl)pyridin-2-yl)-9H-carbazole (11)

Off white solid, 208 mg, 80% yield; mp =128-130° C.; Rƒ=0.30 (Hexane/EtOAc=95/05); ¹H NMR (700 MHZ, CDCl₃) δ=8.16-8.10 (m, 3 H), 8.04 (t, J=7.7 Hz, 1 H), 7.97 (d, J=8.2 Hz, 3 H), 7.73 (d, J=7.7 Hz, 1 H), 7.65 (d, J=7.7 Hz, 1 H), 7.52-7.48 (m, 1 H), 7.46-7.41 (m, 5 H), 7.35-7.30 (m, 5 H); ¹³C NMR (175 MHz, CDCl₃) δ=157.7, 151.4, 141.3, 139.6, 138.1, 133.4, 131.4, 129.9, 128.8, 128.5, 128.4, 128.4, 126.1, 124.3, 123.2, 121.7, 121.3, 120.8, 120.1, 117.5, 111.4, 93.0, 89.2; HRMS (ESI) calcd for C₃₁H₂₀N₂⁺ (M+Na)⁺ 443.1519, found 443.1521.

2-(5-(tert-butyl)-2-(phenylethynyl)phenyl)-6-(4-methoxyphenyl)pyridine (In)

White solid, 240 mg, 76% yield; mp=116-118° C.; Rƒ=0.40 (Hexane/EtOAc=95/05); ¹H NMR (700 MHZ, CDCl₃) δ=8.14-8.11 (m, 2 H), 7.92 (d, J=1.9 Hz, 1 H), 7.87 (dd, J=0.9, 7.7 Hz, 1 H), 7.82 (t, J=7.7 Hz, 1 H), 7.71-7.68 (m, 1 H), 7.63 (d, J=8.2 Hz, 1 H), 7.45 (dd, J=2.0, 8.1 Hz, 1 H), 7.40-7.35 (m, 2 H), 7.28 (dd, J=2.7, 3.8 Hz, 3 H), 7.02-6.97 (m, 2 H), 3.87 (s, 3 H), 1.41 (s, 9 H); ¹³C NMR (175 MHZ, CDCl_{3) δ=}160.4, 157.7, 156.4, 151.8, 142.1, 136.3, 133.0, 132.2, 131.4, 128.3, 128.2, 127.9, 126.9, 125.4, 123.7, 121.9, 118.6, 117.9, 114.0, 91.7, 89.6, 55.3, 34.9, 31.2; HRMS (ESI) calcd for C₃₀H₂₇NO (M+H)⁺ 418.2165, found 418.2180.

2-(4-methoxyphenyl)-6-(4-(phenylethynyl)-[1,1′-biphenyl]-3-yl)pyridine (1o)

White solid, 285 mg, 86% yield; mp=113-115° C.; Rƒ=0.40 (Hexane/EtOAc=95/05); ¹H NMR (700 MHZ, CDCl₃) δ=8.15 (s, 1 H), 8.13 (d, J=8.2 Hz, 2 H), 7.93 (d, J=7.7 Hz, 1 H), 7.84 (t, J=7.7 Hz, 1 H), 7.77 (d, J=8.0 Hz, 1 H), 7.75-7.69 (m, 3 H), 7.66 (d, J=8.0 Hz, 1 H), 7.49 (t, J=7.3 Hz, 2 H), 7.42-7.37 (m, 3 H), 7.31 (br. s., 3 H), 6.99 (d, J=8.2 Hz, 2 H), 3.87 (s, 3 H); ¹³C NMR (175 MHZ, CDCl₃) δ=160.4, 157.2, 156.6, 142.9, 141.3, 140.3, 136.4, 133.7, 132.1, 131.4, 128.8, 128.6, 128.3, 128.3, 128.1, 127.7, 127.2, 126.7, 123.5, 122.0, 120.4, 118.2, 114.0, 93.1, 89.4, 55.3; HRMS (ESI) calcd for C₃₂H₂₃NO (M+H)⁺ 438.1856, found 438.1857.

2-(4-methoxyphenyl)-6-(5-(naphthalen-1-yl)-2-(phenylethynyl)phenyl)pyridine (1p)

White solid, 226 mg, 83% yield; mp=108-110° C.; Rƒ=0.50 (Hexane/EtOAc=95/05); ¹H NMR (700 MHZ, CDCl₃) δ=8.11-8.05 (m, 4 H), 7.94 (d, J=8.0 Hz, 1 H), 7.97 (d, J=7.7 Hz, 1 H), 7.91 (d, J=8.0 Hz, 1 H), 7.86-7.82 (m, 2 H), 7.71 (d, J=7.7 Hz, 1 H), 7.60-7.52 (m, 4 H), 7.51-7.47 (m, 1 H), 7.44 (br. s., 2 H), 7.32 (br. s., 3 H), 6.95 (d, J=7.7 Hz, 2 H), 3.84 (s, 3 H); ¹³C NMR (175 MHz, CDCl₃) δ=160.4, 157.0, 156.5, 142.4, 141.0, 139.4, 136.5, 133.8, 133.3, 132.0, 131.6, 131.5, 131.4, 129.9, 128.3, 128.3, 128.2, 128.0, 127.0, 126.2, 126.0, 125.9, 125.4, 123.5, 121.9, 120.4, 118.1, 114.0, 92.9, 89.5, 55.3; HRMS (ESI) calcd for C₃₆H₂₅NO (M+H)⁺ 488.2009, found 488.2000.

### Example 3

### Optimization of reaction conditions^{a}

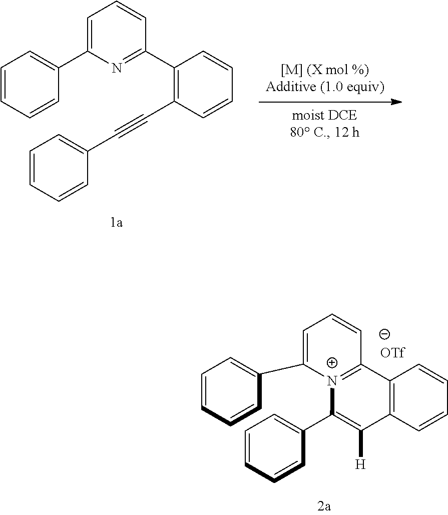

| Sr. | | | Yield |
|---|---|---|---|
| No. | [M] (X mol %) | Additive | (%)^{[b]} |
| 1. | Ph₃PAuNTf₂ (10 mol %) | NaOTf | — |
| 2. | (C₆F₅)₃PAuNTf₂ (10 mol %) | NaOTf | — |
| 3. | JohnPhosAuNTf₂ (10 mol %) | NaOTf | — |
| 4. | IPrAuNTf₂ (10 mol %) | NaOTf | — |
| 5. | AgOTf (10 mol %) | NaOTf | 08 |
| 6. | AgOTf (10 mol %) | — | 08 |
| 7. | AgOTf (25 mol %) | — | 19 |
| 8. | AgOTf (50 mol %) | — | 42 |
| 8. | AgOTf (100 mol %) | — | 80 |

^{[a]}Reaction conditions: 0.15 mmol 1a, cat [M], moist DCE, 80° C., 12 h.
^{[b]}Isolated yields. (moist DCE was prepared with 5 ml of dry DCE and 0.300 mmol of water)

### Example 4

### Silver-Promoted Hydroamination of pyridiono-alkynes

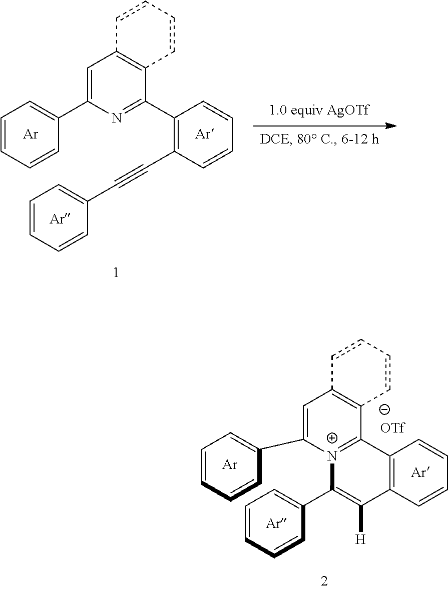

Representative procedure: To a screw-cap vial containing a stir bar were added 2-phenyl-6-(2-(phenylethynyl)phenyl)pyridine (1a) (50 mg, 0.15 mmol, 1.0 equiv), AgOTf (39mg, 0.15 mmol, 1.0 equiv) and DCE (5 mL). The reaction vial was fitted with a cap, evacuated and backfilled with N₂ and heated at 80° C. for 6-12 h. When the reaction time was completed, the reaction mixture was allowed to cool at ambient temperature. The mixture was diluted with CH₂Cl₂ (10 mL) and the combined mixture was concentrated in vacuo and the resulting residue was purified by column chromatography on silica (CH₂Cl₂/MeOH; 95:05) to afford the product 2a in 85% yield.

4,6-diphenylpyrido[2,1-a]isoquinolin-5-ium trifluoromethanesulfonate (2a)

Yellow solid, 61 mg, 85% yield; mp =210-212° C.; Rƒ=0.50 (CH₂C₁₂/MeOH=95/05); ¹H NMR (500 MHZ, DMSO-d₆) δ=9.62 (dd, J=1.1, 8.8 Hz, 1 H), 9.23 (d, J=8.4 Hz, 1 H), 8.74 (dd, J=7.5, 8.5 Hz, 1 H), 8.29 (d, J=7.2 Hz, 1 H), 8.25 (s, 1 H), 8.19-8.15 (m, 1 H), 8.14-8.08 (m, 2 H), 7.32-7.28 (m, 2 H), 7.27-7.22 (m, 1 H), 7.22-7.18 (m, 2 H), 7.18-7.12 (m, 5 H); ¹³C NMR (125 MHz, DMSO-d₆) δ=150.8, 148.3, 141.2, 139.9, 137.0, 136.8, 134.2, 131.7, 130.9, 129.8, 128.6, 128.5, 128.4, 128.3, 128.3, 128.1, 127.5, 127.4, 125.8, 125.3, 122.3, 120.6; HRMS (ESI) calcd for C₂₅H₁₈N⁺ (M-OTf)⁺332.1434, found 332.1421.

6-phenyl-4-(p-tolyl)pyrido[2,1-a]isoquinolin-5-iumtrifluoromethanesulfonate (2b)

Yellow solid, 62 mg, 86% yield; mp=238-240° C.; Rƒ=0.50 (CH₂C₁₂/MeOH=95/05); ¹H NMR (500 MHZ, CD₃CN) δ=9.28 (d, J=8.7 Hz, 1 H), 8.96 (d, J=8.4 Hz, 1 H), 8.60 (dd, J=7.6, 8.4 Hz, 1 H), 8.22-8.14 (m, 1 H), 8.14-8.09 (m, 1 H), 8.09-8.04 (m, 1 H), 7.98 (s, 1 H), 7.94 (dd, J=1.3, 7.4 Hz, 1 H), 7.20-7.12 (m, 3 H), 7.12-7.03 (m, 4 H), 7.00 (d, J=8.1 Hz, 2 H), 2.24 (s, 3 H); ¹³C NMR (125 MHZ, CD₃CN) δ=153.2, 149.6, 142.8, 142.0, 141.0, 138.0, 135.7, 135.1, 132.9, 132.4, 132.1, 130.4, 130.0, 129.7, 129.6, 129.5, 129.5, 129.3, 128.9, 128.7, 126.7, 126.4, 123.0, 122.8, 122.5, 118.7, 21.4, 153.2, 149.6, 142.8, 142.0, 141.0, 138.0, 135.7, 135.1, 132.9, 132.4, 132.1, 130.4, 130.0, 129.7, 129.6, 129.5, 129.5, 129.3, 128.9, 128.7, 126.7, 126.4, 123.0, 122.8, 122.5, 118.7, 21.4, HRMS (ESI) calcd for C₂₆H₂₀N⁺ (M-OTf)⁺ 346.1590, found 346.1614.

4-([1.1′-biphenyl]-4-yl)-6-phenylpyrido[2,1-a]isoquinolin-5-ium trifluoro methanesulfonate (2c)

Yellow solid, 56 mg, 82% yield; mp=203-205° C.; Rƒ=0.50 (CH₂C₁₂/MeOH=95/05); ¹H NMR (500 MHZ, CD₃CN) δ=9.32 (dd, J=1.1, 8.6 Hz, 1 H), 8.99 (d, J=8.2 Hz, 1 H), 8.63 (dd, J=7.6, 8.5 Hz, 1 H), 8.23-8.17 (m, 1 H), 8.16-8.11 (m, 1 H), 8.11-8.06 (m, 1 H), 8.05-7.99 (m, 2 H), 7.59-7.53 (m, 2 H), 7.52-7.45 (m, 2 H), 7.45-7.39 (m, 3 H), 7.31-7.24 (m, 2 H), 7.17-7.11 (m, 5 H); ¹³C NMR (125 MHZ, CD₃CN) δ=152.5, 149.6, 143.6, 142.7, 140.8, 140.5, 138.0, 136.7, 135.6, 135.6, 135.6, 132.9, 132.1, 130.1, 130.0, 129.5, 129.2, 128.9, 128.8, 128.2, 128.0, 126.5, 126.4, 123.2, 123.1; HRMS (ESI) calcd for C₃₁H₂₂N⁺ (M-OTf)⁺ 408.1747, found 408.1756.

4-(4-cyanophenyl)-6-phenylpyrido[2,1-a]isoquinolin-5-ium trifluoromethane sulfonate (2d)

Yellow solid, 50 mg, 71% yield; mp=232-234° C.; Rƒ=0.40 (CH₂C₁₂/MeOH=95/05); ¹H NMR (500 MHZ, CD₃CN) δ=9.37 (d, J=8.5 Hz, 1 H), 9.02 (d, J=8.2 Hz, 1 H), 8.64 (t, J=8.0 Hz, 1 H), 8.26-8.20 (m, 1 H), 8.17 (t, J=7.2 Hz, 1 H), 8.15-8.06 (m, 2 H), 7.96 (d, J=6.9 Hz, 1 H), 7.60-7.47 (m, J=8.4 Hz, 2 H), 7.40-7.30 (m, J=8.2 Hz, 2 H), 7.27-7.17 (m, 3 H), 7.13 (d, J=7.0 Hz, 2 H); ¹³C NMR (175 MHz, CD₃CN) δ=150.1, 149.8, 142.2, 141.5, 140.8, 137.7, 135.9, 133.5, 133.1, 132.4, 130.4, 130.1, 130.0, 129.9, 129.5, 129.4, 129.1, 126.6, 126.4, 124.1, 122.13, 118.8, 114.0; HRMS (ESI) calcd for C₂₆H₁₇N⁺ (M-OTf)⁺ 357.1386, found 357.1407.

4-(4-acetylphenyl)-6-phenylpyrido[2,1-a]isoquinolin-5-ium trifluoromethane sulfonate (2e)

Greenish solid, 49 mg, 70% yield; mp=236-238° C.; Rƒ=0.20 (CH₂Cl₂/MeOH=95/05); ¹H NMR (500 MHZ, CD₃CN) δ=9.36 (d, J=8.5 Hz, 1 H), 9.01 (d, J=8.2 Hz, 1 H), 8.63 (t, J=7.9 Hz, 1 H), 8.25-8.18 (m, 1 H), 8.16 (t, J=7.4 Hz, 1 H), 8.13-8.08 (m, 1 H), 8.06 (s, 1 H), 7.98 (d, J=7.3 Hz, 1 H), 7.76-7.68 (m, J=7.9 Hz, 2 H), 7.35-7.28 (m, J=8.1 Hz, 2 H), 7.19-7.09 (m, 5 H), 2.51 (s, 3 H); ¹³C NMR (125 MHZ, CD₃CN) δ=198.2, 151.3, 149.7, 142.5, 141.4, 140.9, 138.6, 137.8, 135.8, 133.1, 132.4, 130.2, 129.8, 129.8, 129.6, 129.4, 129.3, 129.1, 129.0, 126.6, 126.4, 123.7, 119.2, 117.0, 27.1; HRMS (ESI) calcd for C₂₇H₂₀ON⁺ (M-OTf)⁺ 374.1539, found 374.1541.

4-(4-methoxyphenyl)-6-phenylpyrido[2,1-a]isoquinolin-5-ium trifluoromethane sulfonate (2f)

Yellow solid, 66 mg, 94% yield; mp=200-202° C.; Rƒ=0.30 (CH₂Cl₂/MeOH=95/05); ¹H NMR (500 MHZ, DMSO-d₆) δ=9.55 (dd, J=1.0, 8.6 Hz, 1 H), 9.19 (d, J=8.2 Hz, 1 H), 8.71 (dd, J=7.6, 8.4 Hz, 1 H), 8.27 (d, J=7.5 Hz, 1 H), 8.20 (s, 1 H), 8.15 (t, J=7.5 Hz, 1 H), 8.12-8.06 (m, 2 H), 7.28-7.24 (m, 2 H), 7.20-7.13 (m, 5 H), 6.79-6.72 (m, 2 H), 3.71 (s, 3 H); ¹³C NMR (125 MHz, DMSO-d₆) δ=160.5, 151.1, 148.1, 141.2, 139.8, 137.0, 134.0, 131.5, 130.8, 130.0, 129.7, 129.4, 128.6, 128.3, 128.2, 128.2, 127.2, 127.2, 125.7, 125.3, 121.7, 120.68, 114.0, 55.4; HRMS (ESI) calcd for C₂₆H₂₀NO⁺ (M-OTf)⁺ 362.1539, found 362.1521.

4-(4-(9H-carbazol-9-yl)phenyl)-6-phenylpyrido[2,1-a]isoquinolin-5-ium trifluoro methane sulfonate (2g)

Yellow solid, 52 mg, 80% yield; mp=230-232° C.; Rƒ=0.30 (CH₂Cl₂/MeOH=95/05); ¹H NMR (700 MHZ, CD₃CN) δ=9.37 (dd, J=0.9, 8.6 Hz, 1 H), 9.02 (d, J=8.4 Hz, 1 H), 8.68 (t, J=8.0 Hz, 1 H), 8.22 (d, J=7.5 Hz, 1 H), 8.19 (d, J=7.7 Hz, 2 H), 8.15 (t, J=7.4 Hz, 1 H), 8.12-8.07 (m, 3 H), 7.53-7.46 (m, 4 H), 7.45-7.41 (m, 2 H), 7.37-7.32 (m, 4 H), 7.31-7.26 (m, 5 H); ¹³C NMR (175 MHZ, CD₃CN) δ=151.7, 149.7, 142.5, 141.2, 140.9, 140.1, 138.1, 136.5, 135.6, 133.0, 132.3, 131.2, 130.3, 130.0, 129.9, 129.4, 129.3, 129.2, 129.0, 127.9, 127.3, 126.6, 126.4, 124.4, 123.4, 122.1, 121.6, 121.5, 110.7; HRMS (ESI) calcd for C₃₇H₂₅N₂⁺ (M-OTf)⁺ 497.2012, found 497.2005.

4-(3,5-dimethoxyphenyl)-6-phenylpyrido[2,1-a]isoquinolin-5-ium trifluoro methane sulfonate (2h)

Yellow solid, 74 mg, 93% yield; mp=178-180° C.; Rƒ=0.30 (CH₂Cl₂/MeOH=93/07); ¹H NMR (500 MHZ, CD₃CN) δ=9.35 (d, J=8.7 Hz, 1 H), 9.02 (d, J=8.2 Hz, 1 H), 8.67-8.61 (m, 1 H), 8.24-8.19 (m, 1 H), 8.18-8.14 (m, 1 H), 8.12 (dd, J=1.3, 8.3 Hz, 1 H), 8.04 (s, 1 H), 8.02 (dd, J=1.3, 7.4 Hz, 1 H), 7.28-7.21 (m, 3 H), 7.20-7.15 (m, 2 H), 6.38 (d, J=2.1 Hz, 2 H), 6.33 (t, J=2.2 Hz, 1 H), 3.69 (s, 6 H); ¹³C NMR (125 MHZ, CD₃CN) δ=162.0, 152.5, 149.5, 142.9, 140.8, 139.4, 137.8, 135.6, 133.1, 132.2, 129.9, 129.5, 129.5, 129.1, 128.9, 126.6, 126.5, 123.4, 122.2, 107.8, 103.3, 56.4; HRMS (ESI) calcd for C₂₇H₂₂O₂N⁺ (M-OTf)⁺ 392.1645, found 392.1623.

4-(anthracen-9-yl)-6-phenylpyrido[2,1-a]isoquinolin-5-ium trifluoromethane sulfonate (2i)

Orangish yellow solid, 50 mg, 74% yield, mp=280-282° C.; Rƒ=0.30 (CH₂Cl₂/MeOH=95/05); ¹H NMR (500 MHZ, CD₃CN) δ=9.61 (d, J=8.7 Hz, 1 H), 9.14 (dd, J=3.4, 5.7 Hz, 1 H), 8.77 (t, J=8.0 Hz, 1 H), 8.48 (s, 1 H), 8.23 (d, J=7.3 Hz, 1 H), 8.20-8.07 (m, 3 H), 8.07-7.96 (m, J=8.4 Hz, 2 H), 7.75 (s, 1 H), 7.52 (t, J=7.5 Hz, 2 H), 7.40 (t, J=7.6 Hz, 2 H), 7.28-7.13 (m, J=8.7 Hz, 2 H), 6.60-6.43 (m, 1 H), 6.37 (t, J=7.6 Hz, 2 H), 6.24 (d, J=7.5 Hz, 2 H); ¹³C NMR (175 MHz, CD₃CN) δ=149.3, 148.6, 141.9, 140.5, 135.7, 135.5, 133.3, 132.7, 132.4, 132.3, 131.8, 130.1, 130.0, 129.7, 129.4, 129.1, 129.0, 128.3, 127.9, 127.4, 127.0, 126.8, 126.7, 124.7, 124.5, 122.1; HRMS (ESI) calcd for C₃₃H₂₂N⁺ (M-OTf)⁺ 432.1725, found 432.1728.

4-(phenanthren-9-yl)-6-phenylpyrido[2,1-a]isoquinolin-5-ium trifluoromethane sulfonate (2j)

Yellow solid, 57 mg, 85% yield; mp=268-270° C.; Rƒ=0.30 (CH₂Cl₂/MeOH=95/05); ¹H NMR (500 MHZ, CD₃CN) δ=9.48 (d, J=8.7 Hz, 1 H), 9.07 (d, J=8.1 Hz, 1 H), 8.71 (t, J=8.1 Hz, 1 H), 8.65 (d, J=8.5 Hz, 1 H), 8.68 (d, J=8.5 Hz, 1 H), 8.22 (dd, J=1.8, 5.6 Hz, 1 H), 8.19-8.08 (m, 3 H), 7.89 (s, 1 H), 7.82-7.73 (m, 2 H), 7.72-7.62 (m, 3 H), 7.52-7.46 (m, 1 H), 7.40 (d, J=8.1 Hz, 1 H), 6.90 (d, J=7.6 Hz, 1 H), 6.65 (t, J=7.6 Hz, 1 H), 6.61-6.50 (m, 2 H), 6.46 (d, J=7.5 Hz, 1 H); ¹³C NMR (125 MHZ, CD₃CN) δ=150.8, 149.2, 142.6, 140.7, 136.8, 135.7, 133.3, 132.8, 132.4, 132.2, 131.8, 131.4, 131.3, 131.2, 130.4, 129.9, 129.5, 129.3, 129.2, 129.0, 128.7, 128.6, 128.6, 128.5, 128.4, 128.3, 127.9, 126.7, 126.7, 125.8, 124.4, 124.0, 123.6, 123.5, 122.2; ); HRMS (ESI) calcd for C₃₃H₂₂N⁺ (M-OTf)⁺ 432.1747, found 432.1750.

4-(benzo[b]thiophen-2-yl)-6-phenylpyrido[2,1-a]isoquinolin-5-ium trifluoro methane sulfonate (2k)

Yellow solid, 62 mg, 90% yield, mp=208-210° C.; Rƒ=0.40 (CH₂Cl₂/MeOH=95/05); ¹H NMR (500 MHZ, CD₃CN) δ=9.32 (dd, J=1.2, 8.7 Hz, 1 H), 8.98 (d, J=8.4 Hz, 1 H), 8.61 (dd, J=7.5, 8.5 Hz, 1 H), 8.22 (d, J=7.3 Hz, 1 H), 8.15 (dt, J=1.1, 7.6 Hz, 2 H), 8.12-8.05 (m, 2 H), 7.85-7.76 (m, 1 H), 7.75-7.69 (m, 1 H), 7.39 (tt, J=5.6, 7.3 Hz, 2 H), 7.35 (s, 1 H), 7.28 (dd, J=1.4, 8.0 Hz, 2 H), 7.12-7.00 (m, 3 H); ¹³C NMR (125 MHZ, CD₃CN) δ=150.0, 146.1, 142.7, 141.7, 140.7, 139.7, 137.5, 137.3, 135.9, 133.2, 132.4, 130.7, 130.2, 129.9, 129.5, 129.5, 129.4, 128.4, 127.6, 126.7, 126.5, 126.3, 126.0, 123.9, 123.5, 122.1; HRMS (ESI) calcd for C₂₇H₁₈NS⁺ (M-OTf)⁺ 388.1154, found 388.1182.

4-(9H-carbazol-9-yl)-6-phenylpyrido[2,1-a]isoquinolin-5-ium trifluoromethane sulfonate (21)

Yellow solid, 57 mg, 85% yield; mp=290-298° C.; Rƒ=0.30 (CH₂Cl₂/MeOH=95/05); ¹H NMR (500 MHZ, CD₃CN) δ=9.44 (d, J=8.7 Hz, 1 H), 9.05 (d, J=8.1 Hz, 1 H), 8.80 (t, J=8.2 Hz, 1 H), 8.38 (d, J=7.8 Hz, 1 H), 8.22-8.10 (m, 3 H), 7.99-7.84 (m, 3 H), 7.43-7.31 (m, 4 H), 7.00 (d, J=8.1 Hz, 2 H), 6.68 (qd, J=4.3, 8.5 Hz, 1 H), 6.60 (d, J=4.4 Hz, 4 H); ¹³C NMR (125 MHZ, CD₃CN) δ=149.0, 143.4, 142.2, 140.6, 139.1, 135.8, 134.6, 132.4, 129.6, 129.4, 129.3, 128.0, 127.6, 127.0, 126.9, 126.7, 125.9, 125.5, 124.0, 123.4, 121.6, 111.6; HRMS (ESI) calcd for C₃₁H₂₂N₂⁺ (M-OTf)⁺ 421.1699, found 421.1697.

4-(4-methoxyphenyl)-10-methyl-6-phenylpyrido[2,1-a]isoquinolin-5-ium trifluoro methanesulfonate (2m)

Yellow solid, 63 mg, 90% yield; mp=190-192° C.; Rƒ=0.30 (CH₂Cl₂/MeOH=95/05); ¹H NMR (400 MHZ, DMSO-d₆) δ=9.53 (d, J=8.5 Hz, 1 H), 9.04 (s, 1 H), 8.68 (t, J=7.9 Hz, 1 H), 8.27-8.12 (m, 2 H), 8.08-7.96 (m, 2 H), 7.30-7.22 (m, J=8.3 Hz, 2 H), 7.21-7.12 (m, 5 H), 6.80-6.70 (m, J=8.5 Hz, 2 H), 3.71 (s, 3 H), 2.71 (s, 3 H); ¹³C NMR (100 MHz, DMSO-d₆) δ=160.4, 150.9, 147.8, 141.3, 140.5, 139.5, 137.0, 135.5, 129.9, 129.6, 129.5, 128.5, 128.2, 128.1, 128.1, 127.2, 127.1, 125.3, 125.1, 121.6, 120.7, 114.0, 55.4, 21.7; HRMS (ESI) calcd for C₂₇H₂₂NO⁺ (M-OTf)⁺ 376.1696, found 376.1692.

10-(tert-butyl)-4-(4-methoxyphenyl)-6-phenylpyrido[2,1-a]isoquinolin-5-ium trifluoro methanesulfonate (2n)

Yellow solid, 62 mg, 92% yield; mp=245-247° C.; Rƒ=0.30 (CH₂Cl₂/MeOH=95/05); ¹H NMR (500 MHZ, CD₃CN) δ=9.35 (dd, J=1.2, 8.7 Hz, 1 H), 8.84 (s, 1 H), 8.55 (dd, J=7.5, 8.5 Hz, 1 H), 8.23 (dd, J=1.7, 8.4 Hz, 1 H), 8.11 (d, J=8.4 Hz, 1 H), 7.94 (s, 1 H), 7.91 (dd, J=1.3, 7.4 Hz, 1 H), 7.26-7.12 (m, 5 H), 7.12-7.06 (m, 2 H), 6.76-6.61 (m, 2 H), 3.74 (s, 3 H), 1.55 (s, 9 H); ¹³C NMR (125 MHZ, CD₃CN) δ=162.2, 156.0, 153.0, 149.5, 142.4, 140.4, 138.1, 134.0, 131.1, 131.0, 130.5, 129.9, 129.6, 129.3, 129.2, 128.5, 128.4, 126.3, 123.5, 122.7, 122.2, 118.7, 115.3, 56.3, 36.8, 31.4; HRMS (ESI) calcd for C₃₀H₂₈ON⁺ (M-OTf)⁺ 418.2165, found 418.2172.

4-(4-methoxyphenyl)-6,10-diphenylpyrido[2,1-a]isoquinolin-5-ium trifluoro methanesulfonate (2o)

Yellow solid, 59 mg, 88% yield; mp=250-252° C.; Rƒ=0.30 (CH₂Cl₂/MeOH=95/05); ¹H NMR (500 MHZ, CD₃CN) δ=9.41 (d, J=8.5 Hz, 1 H), 9.12 (s, 1 H), 8.59 (t, J=8.0 Hz, 1 H), 8.40-8.35 (m, 1 H), 8.27-8.21 (m, 1 H), 8.01-7.98 (m, 1 H), 7.98-7.93 (m, 3 H), 7.63 (t, J=6.9 Hz, 2 H), 7.58-7.51 (m, 1 H), 7.22-7.16 (m, 5 H), 7.15-7.10 (m, 2 H), 6.72 (d, J=8.9 Hz, 2 H), 3.75 (s, 3 H); ¹³C NMR (125 MHZ, CD₃CN) δ=162.3, 157.5, 153.2, 149.6, 144.6, 142.8, 140.7, 140.1, 138.0, 134.5, 131.9, 131.2, 130.5, 130.4, 130.0, 130.0, 129.6, 129.6, 128.9, 128.5, 128.4, 127.0, 124.3, 122.9, 122.2, 115.3, 56.3; HRMS (ESI) calcd for C₃₂H₂₄NO⁺ (M-OTf)⁺ 438.1852, found 438.1825.

4-(4-methoxyphenyl)-10-(naphthalen-1-yl)-6-phenylpyrido[2,1-a]isoquinolin-5-ium trifluoromethanesulfonate (2p)

Yellow solid, 59 mg, 91% yield; mp=221-223° C.; Rƒ=0.30 (CH₂Cl₂/MeOH=95/05); ¹H NMR (500 MHZ, CD₃CN) δ=9.29-9.24 (m, 1 H), 9.05 (s, 1 H), 8.54 (dd, J=7.6, 8.4 Hz, 1 H), 8.29 (d, J=8.2 Hz, 1 H), 8.23 (dd, J=1.4, 8.1 Hz, 1 H), 8.11-8.04 (m, 3 H), 7.95 (dd, J=1.1, 7.4 Hz, 1 H), 7.88 (d, J=8.4 Hz, 1 H), 7.73-7.67 (m, 2 H), 7.65-7.60 (m, 1 H), 7.56 (ddd, J=1.1, 6.9, 8.3 Hz, 1 H), 7.24-7.18 (m, 5 H), 7.18-7.14 (m, 2 H), 6.78-6.70 (m, 2 H), 3.76 (s, 3 H); ¹³C NMR (125 MHZ, CD₃CN) δ=162.3, 153.2, 149.5, 144.5, 142.9, 140.7, 139.0, 138.0, 137.3, 134.9, 132.1, 131.9, 131.1, 130.4, 130.0, 129.7, 129.6, 129.3, 129.0, 128.4, 128.0, 127.4, 127.3, 126.7, 126.7, 126.0, 122.7, 122.1, 115.3, 56.3; HRMS (ESI) calcd for C₃₆H₂₆ON⁺ (M-OTf)⁺ 489.2042, found 489.2070.

10-chloro-4-(4-methoxyphenyl)-6-phenylpyrido[2,1-a]isoquinolin-5-ium trifluoro methanesulfonate (2q)

Yellow solid, 60 mg, 87% yield; mp=227-229° C. Rƒ=0.30 (CH₂Cl₂/MeOH=95/05); ¹H NMR (500 MHZ, CD₃CN) δ=9.21 (dd, J=1.2, 8.5 Hz, 1 H), 8.98 (d, J=1.5 Hz, 1 H), 8.61 (dd, J=7.6, 8.4 Hz, 1 H), 8.14 (d, J=8.4 Hz, 1 H), 8.06 (dd, J=1.9, 8.5 Hz, 1 H), 7.98 (dd, J=1.4, 7.5 Hz, 1 H), 7.95 (s, 1 H), 3.74 (s, 3 H); ¹³C NMR (125 MHZ, CD₃CN) δ=162.3, 153.4, 148.4, 143.1, 141.1, 137.7, 137.6, 135.5, 131.3, 131.2, 131.1, 130.2, 130.1, 130.1, 129.5, 128.4, 128.0, 127.7, 125.9, 122.8, 122.2, 115.3, 56.3; HRMS (ESI) calcd for C₂₆H₁₉ClON⁺ (M-OTf)⁺ 396.1150, found 396.1130.

4-(4-methoxyphenyl)-9-methyl-6-phenylpyrido[2,1-a]isoquinolin-5-ium trifluoro methanesulfonate (2r)

Yellow solid, 59 mg, 85% yield; mp=190-192° C., Rƒ=0.30 (CH₂Cl₂/MeOH=95/05); ¹H NMR (500 MHZ, CD₃CN) δ=9.17 (d, J=8.5 Hz, 1 H), 8.82 (d, J=8.7 Hz, 1 H), 8.53 (dd, J=7.6, 8.5 Hz, 1 H), 7.95 (s, 1 H), 7.92-7.85 (m, 3 H), 7.19-7.14 (m, 5 H), 7.12-7.08 (m, 2 H), 6.74-6.68 (m, 2 H), 3.74 (s, 3 H), 2.67 (s, 3 H); ¹³C NMR (125 MHZ, CD₃CN) δ=162.2, 152.7, 149.6, 147.3, 143.0, 140.5, 138.1, 133.9, 133.1, 131.1, 130.5, 130.0, 129.6, 128.9, 128.8, 128.5, 128.4, 126.5, 124.3, 122.3, 122.2, 115.3, 56.3, 22.1; HRMS (ESI) calcd for C₂₇H₂₂ON⁺ (M-OTf)⁺ 376.1696, found 376.1672.

9-chloro-4-(4-methoxyphenyl)-6-phenylpyrido[2,1-a]isoquinolin-5-ium trifluoro methanesulfonate (2s)

Yellow solid, 55 mg, 80% yield; mp=219-221° C., Rƒ=0.30 (CH₂Cl₂/MeOH=95/05); ¹H NMR (500 MHZ, CD₃CN) δ=9.18 (dd, J=0.9, 8.5 Hz, 1 H), 8.90 (d, J=9.0 Hz, 1 H), 8.59 (dd, J=7.6, 8.5 Hz, 1 H), 8.17 (d, J=2.1 Hz, 1 H), 8.02 (dd, J=2.1, 8.9 Hz, 1 H), 7.95 (dd, J=1.2, 7.5 Hz, 1 H), 7.88 (s, 1 H), 7.20-7.15 (m, 5 H), 7.12-7.09 (m, 2 H), 6.74-6.70 (m, 2 H), 3.74 (s, 3 H); ¹³C NMR (125 MHZ, CD₃CN) δ=162.4, 153.4, 151.9, 149.2, 144.0, 141.3, 141.1, 137.7, 134.0, 132.4, 131.2, 130.2, 129.7, 129.6, 128.6, 128.4, 127.5, 125.1, 122.6, 115.3, 56.4; HRMS (ESI) calcd for C₂₆H₁₉ClON⁺ (M-OTf)⁺ 396.1150, found 396.1128.

4-(4-methoxyphenyl)-8.10-dimethyl-6-phenylpyrido[2,1-a]isoquinolin-5-ium trifluoro methanesulfonate (2t)

Yellow solid, 59 mg, 85% yield; mp=214-216° C., Rƒ=0.40 (CH₂Cl₂/MeOH=95/05); ¹H NMR (500 MHZ, CD₃CN) δ=9.21 (d, J=8.5 Hz, 1 H), 8.63 (s, 1 H), 8.53 (t, J=8.1 Hz, 1 H), 7.93 (s, 1 H), 7.89 (dd, J=1.1, 7.4 Hz, 1 H), 7.81 (s, 1 H), 7.20-7.10 (m, 7 H), 6.71 (d, J=8.7 Hz, 2 H), 3.74 (s, 3 H), 2.74 (s, 3 H), 2.68 (s, 3 H); ¹³C NMR (125 MHZ, CD₃CN) δ=162.1, 152.6, 149.4, 142.9, 141.8, 140.3, 138.3, 137.8, 137.3, 131.0, 130.4, 129.9, 129.8, 129.5, 129.0, 128.6, 126.8, 125.6, 123.7, 122.6, 122.1, 115.2, 56.3, 22.1, 19.0; HRMS (ESI) calcd for C₂₈H₂₄NO⁺ (M-OTf)⁺ 390.1852, found 390.1864.

8,10-dimethoxy-4-(4-methoxyphenyl)-6-phenylpyrido[2,1-a]isoquinolin-5-ium trifluoromethanesulfonate (2u)

Yellow solid, 60 mg, 88% yield; mp=217-219° C.; Rƒ=0.50 (CH₂Cl₂/MeOH=90/10); ¹H NMR (500 MHZ, CD₃CN) δ=9.25-9.19 (m, 1 H), 8.51 (dd, J=7.6, 8.5 Hz, 1 H), 8.01 (s, 1 H), 7.90 (dd, J=1.2, 7.5 Hz, 1 H), 7.80 (d, J=1.7 Hz, 1 H), 7.18-7.11 (m, 6 H), 7.09-7.05 (m, 2 H), 6.73-6.67 (m, 2 H), 4.12 (s, 3 H), 4.07 (s, 3 H), 3.74 (s, 3 H); ¹³C NMR (125 MHZ, CD₃CN) δ=164.0, 162.2, 158.1, 152.6, 148.3, 140.5, 139.8, 138.4, 131.1, 130.6, 129.7, 129.6, 129.5, 128.8, 128.5, 123.3, 123.1, 122.2, 119.4, 115.2, 105.5, 105.5, 98.7, 57.6, 57.4, 56.3; HRMS (ESI) calcd for C₂₈H₂₄O₃N⁺ (M-OTf)⁺ 422.1751, found 422.1760.

4-(4-methoxyphenyl)-6-(p-tolyl)pyrido[2,1-a]isoquinolin-5-ium trifluoromethanesulfonate (2v)

Yellow solid, 57 mg, 82% yield;; mp=257-259° C.; Rƒ=0.30 (CH₂Cl₂/MeOH=95/05); ¹H NMR (500 MHZ, CD₃CN) δ=9.22 (d, J=8.7 Hz, 1 H), 8.93 (d, J=8.4 Hz, 1 H), 8.56 (t, J=8.0 Hz, 1 H), 8.19-8.12 (m, 1 H), 8.10 (t, J=7.5 Hz, 1 H), 8.07-8.01 (m, 1 H), 7.94 (s, 1 H), 7.91 (dd, J=1.1, 7.4 Hz, 1 H), 7.17-7.09 (m, 2 H), 6.98 (s, 4 H), 6.76-6.68 (m, 2 H), 3.75 (s, 3 H), 2.23 (s, 3 H); ¹³C NMR (125 MHZ, CD₃CN) δ=162.2, 153.1, 149.5, 143.0, 140.6, 140.3, 135.3, 135.2, 132.9, 132.0, 131.0, 130.4, 130.0, 129.3, 129.3, 128.4, 128.2, 126.4, 126.3, 123.5, 122.4, 115.1, 56.3, 21.2; HRMS (ESI) calcd for C₂₇H₂₂ON⁺ (M-OTf)⁺ 376.1696, found 376.1677.

6-(2-methoxyphenyl)-4-(4-methoxyphenyl)pyrido[2,1-a]isoquinolin-5-ium trifluoro methanesulfonate (2w)

Yellow solid, 63 mg, 92% yield; mp=278-280° C.; Rƒ=0.30 (CH₂Cl₂/MeOH=95/05); ¹H NMR (500 MHZ, CD₃CN) δ=9.25 (dd, J=1.3, 8.6 Hz, 1 H), 8.95 (d, J=8.2 Hz, 1 H), 8.57 (dd, J=7.6, 8.4 Hz, 1 H), 8.19-8.13 (m, 1 H), 8.10 (dt, J=1.1, 7.4 Hz, 1 H), 8.08-8.03 (m, 1 H), 8.00 (s, 1 H), 7.94 (dd, J=1.4, 7.5 Hz, 1 H), 7.31-7.24 (m, 1 H), 7.22-7.15 (m, 2 H), 7.09-6.96 (m, 1 H), 6.85-6.78 (m, 1 H), 6.75-6.65 (m, 3 H); ¹³C NMR (125 MHz, CD₃CN) δ=162.4, 156.0, 153.1, 148.0, 140.5, 140.4, 135.2, 132.6, 132.2, 132.1, 131.7, 131.1, 130.4, 129.5, 129.3, 129.1, 128.6, 126.6, 126.2, 125.7, 122.6, 122.2, 121.8, 115.4, 114.9, 111.3, 56.3, 56.1; HRMS (ESI) calcd for C₂₇H₂₂O₂N⁺ (M-OTf)⁺ 392.1645, found 392.1675.

6-(4-chlorophenyl)-4-(4-methoxyphenyl)pyrido[2,1-a]isoquinolin-5-ium trifluoro methanesulfonate (2x)

Yellow solid, 55 mg, 80% yield; mp=218-220° C.; Rƒ=0.30 (CH₂Cl₂/MeOH=95/05); ¹H NMR (500 MHz, CD₃CN) δ=9.29-9.20 (m, 1 H), 8.95 (d, J=8.2 Hz, 1 H), 8.67-8.51 (m, 1 H), 8.20-8.14 (m, 1 H), 8.14-8.09 (m, 1 H), 8.09-8.04 (m, 1 H), 7.97 (s, 1 H), 7.94 (dd, J=1.4, 7.5 Hz, 1 H), 7.24-7.12 (m, 4 H), 7.12-7.05 (m, 2 H), 6.82-6.70 (m, 2 H), 3.78 (s, 3 H): ¹³C NMR (125 MHZ, CD₃CN) δ=162.4, 152.8, 149.5, 141.5, 140.9, 136.6, 135.5, 135.2, 132.6, 132.3, 131.2, 130.3, 130.2, 129.5, 129.5, 129.4, 128.8, 126.5, 122.6, 122.1, 115.3, 56.4; HRMS (ESI) calcd for C₂₆H₁₉ClNO⁺ (M-OTf)⁺ 396.1150, found 396.1136.

6-(3,5-dimethoxyphenyl)-4-(4-methoxyphenyl)pyrido[2,1-a]isoquinolin-5-ium trifluoromethanesulfonate (2y)

Yellow solid, 62 mg, 91% yield; mp=222-224° C.; Rƒ=0.30 (CH₂Cl₂/MeOH=97/03); ¹H NMR (500 MHZ, CD₃CN) δ=9.24 (dd, J=1.3, 8.6 Hz, 1 H), 8.95 (d, J=8.2 Hz, 1 H), 8.57 (dd, J=7.5, 8.5 Hz, 1 H), 8.18-8.14 (m, 1 H), 8.11 (dt, J=1.1, 7.5 Hz, 1 H), 8.09-8.04 (m, 1 H), 8.01 (s, 1 H), 7.93 (dd, J=1.4, 7.5 Hz, 1 H), 7.21-7.15 (m, 2 H), 6.81-6.73 (m, 2 H), 6.29-6.19 (m, 3 H), 3.77 (s, 3 H), 3.67 (s, 6 H); ¹³C NMR (125 MHZ, CD₃CN) δ=162.2, 161.9, 153.2, 149.4, 142.5, 140.9, 139.7, 135.5, 132.7, 132.3, 131.4, 130.3, 129.4, 129.4, 128.3, 126.6, 126.5, 123.5, 122.6, 115.1, 107.2, 102.0, 56.4, 56.3; HRMS (ESI) calcd for C₂₈H₂₄O₃N⁺ (M-OTf)⁺ 422.1751, found 422.1775.

6-(2-chloro-5-methoxyphenyl)-4-(4-methoxyphenyl)pyrido[2,1-a]isoquinolin-5-ium trifluoromethanesulfonate (2z)

Yellow solid, 62 mg, 92% yield; mp=175-177° C., Rƒ=0.30 (CH₂Cl₂/MeOH=94/06); ¹H NMR (500 MHZ, CD₃CN) δ=9.31 (d, J=8.5 Hz, 1 H), 8.98 (d, J=7.9 Hz, 1 H), 8.60 (t, J=8.1 Hz, 1 H), 8.22-8.17 (m, 1 H), 8.16-8.09 (m, 2 H), 8.05 (s, 1 H), 7.99 (dd, J=1.2, 7.5 Hz, 1 H), 7.27 (dd, J=2.4, 8.6 Hz, 1 H), 7.13 (dd, J=2.4, 8.6 Hz, 1 H), 7.09-7.02 (m, 1 H), 6.83 (dd, J=2.6, 8.7 Hz, 1 H), 6.79-6.68 (m, 3 H), 3.78 (s, 3 H), 3.73 (s, 3 H); ¹³C NMR (125 MHZ, CD₃CN) δ=162.6, 159.5, 152.7, 148.4, 141.0, 139.6, 136.2, 135.6, 132.8, 132.3, 131.5, 131.2, 130.7, 130.3, 129.8, 129.6, 129.3, 126.8, 126.5, 124.0, 123.3, 118.0, 116.0, 114.9, 56.6, 56.4; HRMS (ESI) calcd for C₂₇H₂₁ClO₂N⁺ (M-OTf)⁺ 426.1255, found 426.1276.

4-(4-methoxyphenyl)-6-(naphthalen-1-yl)pyrido[2,1-a]isoquinolin-5-ium trifluoromethanesulfonate (2aa)

Yellow solid, 60 mg, 88% yield; mp=181-183° C.; Rƒ=0.30 (CH₂Cl₂/MeOH=95/05); ¹H NMR (500 MHZ, CD₃CN) δ=9.35 (d, J=8.7 Hz, 1 H), 9.03 (d, J=8.1 Hz, 1 H), 8.57 (t, J=8.0 Hz, 1 H), 8.22-8.17 (m, 2 H), 8.17-8.09 (m, 2 H), 7.83 (d, J=8.2 Hz, 1 H), 7.79 (d, J=7.3 Hz, 1 H), 7.75 (d, J=8.2 Hz, 1 H), 7.52 (t, J=7.5 Hz, 1 H), 7.40 (t, J=7.6 Hz, 1 H), 7.34-7.28 (m, 2 H), 7.27-7.23 (m, 1 H), 6.94 (dd, J=2.4, 8.6 Hz, 1 H), 6.60-6.48 (m, 2 H), 6.15 (dd, J=2.6, 8.5 Hz, 1 H), 3.54 (s, 3 H); ¹³C NMR (125 MHZ, CD₃CN) δ=161.5, 153.0, 149.2, 140.9, 140.5, 135.5, 134.6, 134.5, 132.7, 132.5, 131.2, 131.2, 130.2, 130.1, 130.0, 129.8, 129.4, 129.1, 128.2, 127.7, 126.7, 126.7, 126.3, 124.8, 123.2, 122.2, 115.4, 114.0, 56.1; HRMS (ESI) calcd for C₃₀H₂₂NO⁺ (M-OTf)⁺ 412.1696, found 412.1711.

8-(4-methoxyphenyl)-6-phenylisoquinolino[3,2-a]isoquinolin-7-ium trifluoro methane sulfonate (2ab)

Yellow solid, 60 mg, 89% yield; Rƒ=0.30 (CH₂Cl₂/MeOH=95/05); ¹H NMR (500 MHz, CD₃CN) δ=9.76 (s, 1 H), 8.91 (d, J=7.9 Hz, 1 H), 8.53 (d, J=8.2 Hz, 1 H), 8.25-8.18 (m, 1 H), 8.08 (d, J=8.9 Hz, 1 H), 8.05-7.96 (m, 3 H), 7.94-7.88 (m, 1 H), 7.65 (s, 1 H), 7.20-7.11 (m, 5 H), 6.94-6.87 (m, 2 H), 6.85-6.80 (m, 2 H), 3.83 (s, 3 H); ¹³C NMR (125 MHZ, CD₃CN) δ=162.6, 156.3, 142.0, 141.5, 139.3, 138.2, 136.8, 133.7, 133.5, 132.7, 131.9, 130.1, 129.9, 129.5, 129.4, 129.3, 129.1, 128.6, 128.4, 128.4, 127.8, 127.3, 127.1, 125.5, 121.2, 115.5, 56.4; HRMS (ESI) calcd for C₂₆H₁₈ON⁺ (M-OTf)⁺412.1705, found 412.1702.

4-methyl-6-phenylpyrido[2,1-a]isoquinolin-5-ium trifluoromethanesulfonate (2ac)

Greenish solid, 47 mg, 60% yield; Rƒ=0.40 (CH₂Cl₂/MeOH=95/05); ¹H NMR (500 MHZ, CD₃CN) δ=9.25 (d, J=8.5 Hz, 1 H), 8.93 (d, J=8.4 Hz, 1 H), 8.51 (t, J=8.1 Hz, 1 H), 8.24-8.16 (m, 2 H), 8.10 (t, J=7.5 Hz, 1 H), 8.08-8.00 (m, 1 H), 7.90 (d, J=7.3 Hz, 1 H), 7.66-7.57 (m, 3 H), 7.57-7.51 (m, 2 H), 2.28 (s, 3 H); ¹³C NMR (125 MHZ, CD₃CN) δ=152.0, 148.7, 141.1, 140.8, 138.0, 135.2, 132.2, 132.0, 131.0, 130.4, 129.6, 129.5, 129.3, 128.4, 126.6, 126.3, 122.5, 120.9, 27.3; HRMS (ESI) calcd for C₂₀H₁₆N⁺ (M-OTf)⁺ 270.1305, found 270.1309.

6-cyclopropyl-4-(4-methoxyphenyl)pyrido[2,1-a]isoquinolin-5-ium trifluoro methanesulfonate (2ad)

Yellow solid, 56 mg, 76% yield; Rƒ=0.40 (CH₂Cl₂/MeOH=95/05); ¹H NMR (500 MHz, CD₃CN) δ=9.17 (d, J=8.4 Hz, 1 H), 8.88 (d, J=8.2 Hz, 1 H), 8.52 (t, J=8.0 Hz, 1 H), 8.13-8.05 (m, 3 H), 8.04 (s, 1 H), 8.02-7.98 (m, 1 H), 7.63-7.54 (m, 2 H), 7.14-7.06 (m, 2 H), 3.89 (s, 3 H), 1.47-1.38 (m, 1 H), 0.73-0.66 (m, 2 H), 0.58-0.51 (m, 2 H); ¹³C NMR (125 MHZ, CD₃CN) δ=163.0, 152.0, 148.8, 145.7, 139.9, 139.8, 135.3, 135.0, 132.9, 131.7, 130.9, 130.7, 130.6, 129.8, 129.5, 128.7, 128.4, 127.9, 127.7, 126.7, 126.3, 126.2, 123.6, 122.8, 122.7, 121.0, 115.6, 56.5, 20.1, 12.0; HRMS (ESI) calcd for C₂₃H₂₀ON⁺ (M-OTf)⁺ 326.1522, found 326.1536.

Variation in counterions:

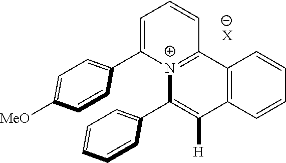

3a, X=NTf₂, 93%
3b, X=SbF₆, 84%
3c, X=BF₄, ₈₈%
3d, X=NO₃, 60%
   4-(4-methoxyphenyl)-6-phenylpyrido[2,1-a]isoquinolin-5-iumbis((trifluoro methyl) sulfonyl) amide (3a)

Yellow solid, 82 mg, 93% yield; Rƒ=0.40 (CH₂Cl₂/MeOH=93/07); ¹H NMR (500 MHz, CD₃CN) δ=9.23 (dd, J=1.2, 8.7 Hz, 1 H), 8.93 (d, J=8.2 Hz, 1 H), 8.57 (dd, J=7.6, 8.5 Hz, 1 H), 8.19-8.14 (m, 1 H), 8.11 (dt, J=1.1, 7.5 Hz, 1 H), 8.08-8.03 (m, 1 H), 7.97 (s, 1 H), 7.93 (dd, J=1.4, 7.5 Hz, 1 H), 7.20-7.14 (m, 5 H), 7.14-7.09 (m, 2 H), 6.75-6.68 (m, 2 H); ¹³C NMR (125 MHz, CD₃CN) δ=162.3, 153.1, 149.6, 142.9, 140.8, 138.0, 135.5, 132.9, 132.2, 131.2, 130.4, 130.0, 129.6, 129.5, 129.4, 128.7, 128.5, 126.5, 126.5, 122.5, 122.3, 119.7, 115.3, 56.4; ¹⁹F NMR (376.5 MHZ, CD₃CN) δ=−80.16.

4-(4-methoxyphenyl)-6-phenylpyrido[2,1-a]isoquinolin-5-ium hexafluorostibate (3b)

Yellow solid, 75 mg, 86% yield; Rƒ=0.50 (CH₂Cl₂/MeOH=93/07); ¹H NMR (500 MHz,, CD₃CN) δ=9.22 (dd, J=1.1, 8.6 Hz, 1 H), 8.93 (d, J=8.2 Hz, 1 H), 8.57 (dd, J=7.6, 8.5 Hz, 1 H), 8.19-8.14 (m, 1 H), 8.13-8.09 (m, 1 H), 8.08-8.03 (m, 1 H), 7.97 (s, 1 H), 7.93 (dd, J=1.2, 7.5 Hz, 1 H), 7.21-7.15 (m, 5 H), 7.14-7.09 (m, 2 H), 6.75-6.68 (m, 2 H), 3.74 (s, 3 H); ¹³C NMR (125 MHZ, CD₃CN) δ=162.3, 153.1, 149.6, 142.9, 140.8, 138.0, 135.5, 132.9, 132.2, 131.2, 130.4, 130.0, 129.6, 129.5, 129.4, 128.7, 128.5, 126.5, 126.5, 122.5, 115.3, 56.4.

4-(4-methoxyphenyl)-6-phenylpyrido[2,1-a]isoquinolin-5-ium tetrafluoroborate (3c)

Yellow solid, 54 mg, 87% yield; Rƒ=0.50 (CH₂Cl₂/MeOH=95/05); ¹H NMR (500 MHz, CD₃CN) δ=9.23 (d, J=8.7 Hz, 1 H), 8.94 (d, J=8.4 Hz, 1 H), 8.58 (t, J=8.0 Hz, 1 H), 8.21-8.13 (m, 1 H), 8.11 (t, J=7.5 Hz, 1 H), 8.08-8.03 (m, 1 H), 7.97 (s, 1 H), 7.93 (dd, J=1.2, 7.5 Hz, 1 H), 7.22-7.13 (m, 5 H), 7.13-7.08 (m, 2 H), 6.72 (d, J=8.7 Hz, 2 H), 3.74 (s, 3 H); ¹³C NMR (125 MHZ, CD₃CN) δ=162.3, 156.6, 153.1, 149.6, 142.9, 140.9, 138.0, 135.6, 135.3, 132.9, 132.3, 132.0, 131.2, 130.4, 130.0, 129.6, 129.4, 129.2, 128.8, 128.6, 128.5, 126.7, 126.5, 126.3, 122.5, 115.3, 56.3; ¹¹B NMR (128.3 MHZ, CD₃CN) δ=−1.16; ¹⁹F NMR (376.5 MHz, CD₃CN) δ=−151.77.

4-(4-methoxyphenyl)-6-phenylpyrido[2,1-a]isoquinolin-5-ium nitrate (3d)

Yellow solid, 35 mg, 60% yield; Rƒ=0.30 (CH₂Cl₂/MeOH=90/10); ¹H NMR (500 MHz, CD₃CN) δ=9.28 (dd, J=1.2, 8.7 Hz, 1 H), 8.97 (d, J=8.2 Hz, 1 H), 8.59 (dd, J=7.6, 8.5 Hz, 1 H), 8.19-8.14 (m, 1 H), 8.13-8.02 (m, 2 H), 7.97 (s, 1 H), 7.94 (dd, J=1.4, 7.5 Hz, 1 H), 7.21-7.14 (m, 5 H), 7.14-7.08 (m, 2 H), 6.76-6.65 (m, 2 H), 3.74 (s, 3 H); ¹³C NMR (125 MHZ, CD₃CN) δ=162.3, 153.1, 149.6, 142.8, 140.9, 138.0, 135.4, 132.9, 132.2, 131.2, 130.4, 130.0, 129.6, 129.5, 129.4, 128.7, 128.5, 126.6, 126.5, 122.6, 115.3, 56.3.

Representative procedure for the synthesis of enyne 2a at 1 mmol scale:

To a screw-cap vial containing a stir bar were added 2-phenyl-6-(2-(phenylethynyl)phenyl)pyridine (1a) (330 mg, 1.0 mmol, 1.0 equiv), AgOTf (256 mg, 1.0 mmol, 1.0 equiv) and DCE (4 mL). The reaction vial was fitted with a cap, evacuated and back filled with N₂ and heated at 80° C. for 6-12 h. When the reaction time was completed, the reaction mixture was allowed to cool at ambient temperature. The mixture was diluted with CH₂Cl₂ (10 mL) and the combined mixture was concentrated in vacuo and the resulting residue was purified by column chromatography on silica (CH₂Cl₂/MeOH; 95:05) to afford the product 2a in 70% yield.

### Photo-physical investigation

### Steady-state absorption and emission studies in solution and solid-state

For solution state photophysical studies (absorption, emission and lifetime) stock for all compounds (1 mM concentration) were prepared in DMSO solvent. For the measurement, the required volume was added from the stock solution in 1 mL solution to make it final concentration as 10 μM. For aggregation induced emission (AIE) measurement, different ratio of DCM-Hexane solvent mixture (0-99%) was prepared by adding required volume of DCM and Hexane, subsequently measurements were performed by maintaining 10 μM dye concentration in each 1 mL solution.

The AIE images were taken in 5 ml vials which were placed on a handled UV (excitation wavelength 365 nm) instrument. For the solid-state absorption and emission measurements, powder samples were prepared by dissolving compound and BaSO₄ in DCM and then evaporated to dryness. Solution Spectra were measured in HORIBA Jobin Yvon Fluorolog using 1 cm path length quartz cuvette. To make the powder more homogenous, mixtures were grinded effectively for few minutes.

For recording of solid-state UV-Vis. spectra, the powder samples were placed on solid-state holder and spectra were recorded in Carry 5000 UV-Vis.-NIR spectrophotometer using diffusion reflectance methods.

To record solid-state fluorescence spectra same powder samples were placed in sample holder and holder was fixed at magic angle (54.7°). Spectra were recorded using same instrument mentioned above.

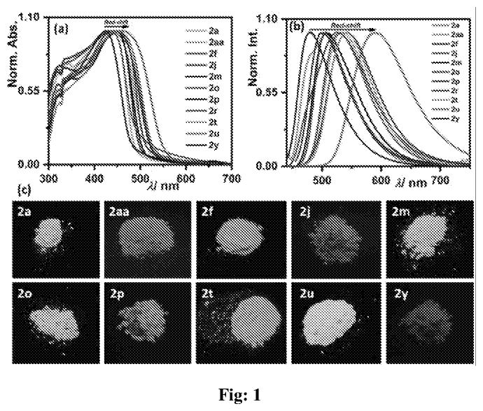

FIG. 1 represents (a) Normalized absorption, and (b) emission spectra of the luminogens herein, in the solid state, (c) Digital fluorescence images of the powder samples of the luminogens collected upon excitation at 365 nm.

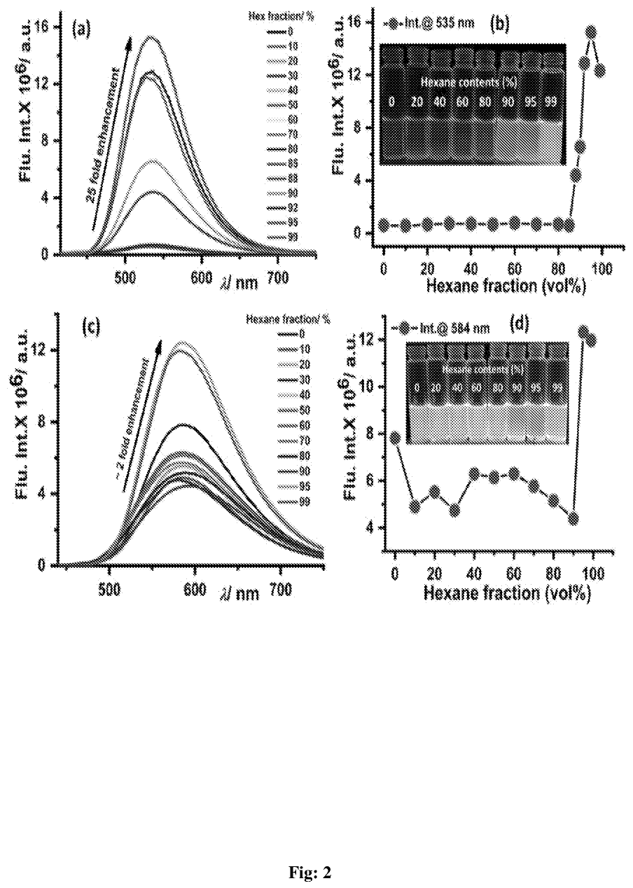

FIG. 2 represents (a), (c) Fluorescence spectra of 2f and 2u in DCM/hexane solvent mixture with increasing fₕₑₓₐₙₑ (0-99%) in DCM. (b), (d) PL intensity change at a certain wavelength with increasing fₕₑₓₐₙₑ (0-99%) in DCM for 2f and 2u respectively; insets of (b) and (d) present the fluorescence images of respective solution with the variation of fₕₑₓₐₙₑ under UV light (λₑₓ=365 nm) exposure.

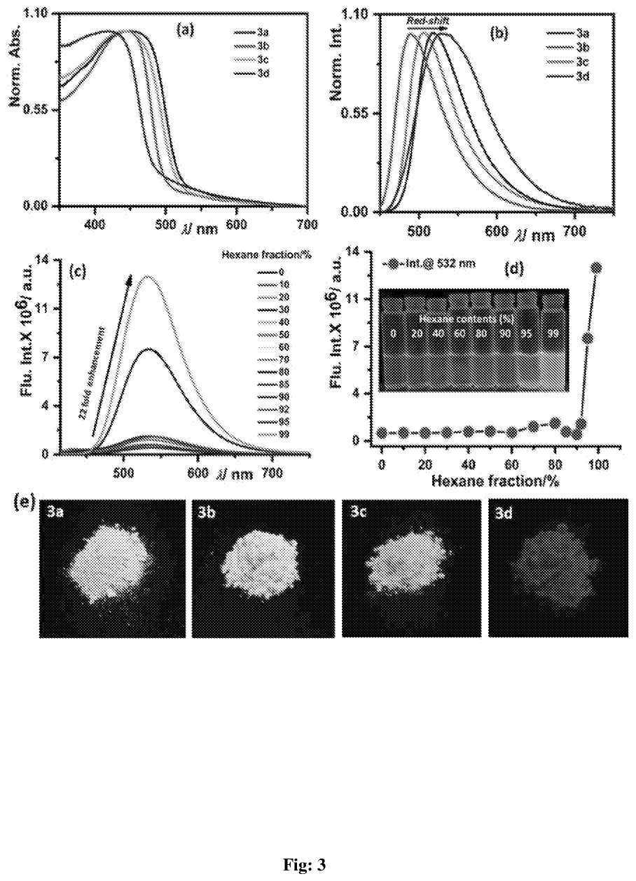

FIG. 3 represents (a) Absorption, and (b) fluorescence spectra of compounds 3a-d containing respective counter-ion mentioned in solid state, (c) Fluorescence spectra of compound 3a in DCM/hexane solvent mixture with increasing fₕₑₓₐₙₑ (0-99%) in DCM. (d) Fluorescence intensity change at a certain wavelength with properties in both solution and the solid state, (e) Digital fluorescence images of the powder samples of luminogens herein, containing different counter-ions collected upon excitation at 365 nm.

Table 1 shows the spectral properties of the luminogens in DMSO at RT under UV light (λ_{exc}=365 nm).

TABLE 1

| | λₘₐₓ | λₘₐₓ | Stokes | Lifetime/ | |
|---|---|---|---|---|---|
| Comp | (abs.)/nm | (em.)/nm | shift/(nm) | ns | Rel. QY |
| 2a | 392 | 508 | 116 | 5.84 | 0.015 |
| 2aa | 397 | 540 | 143 | 4.09 | 0.062 |
| 2f | 404 | 540 | 136 | 5.32 | 0.018 |
| 2j | 397 | 544 | 147 | 5.19 | 0.220 |
| 2m | 409 | 540 | 131 | 3.6 | 0.020 |
| 2o | 417 | 546 | 129 | 3.72 | 0.036 |
| 2p | 412 | 552 | 140 | 3.38 | 0.032 |
| 2r | 405 | 538 | 133 | 5.93 | 0.017 |
| 2t | 410 | 542 | 132 | 4.49 | 0.024 |
| 2u | 430 | 602 | 172 | 4.2 | 0.125 |
| 2y | 401 | 540 | 139 | 5.72 | 0.006 |

The photophysical studies in the solid state as well as in solution reveal that the electronic, as well as steric nature of substituents and counter-ions, has a remarkable effect on the photophysical properties of these compounds.

### Cytotoxicity Assay

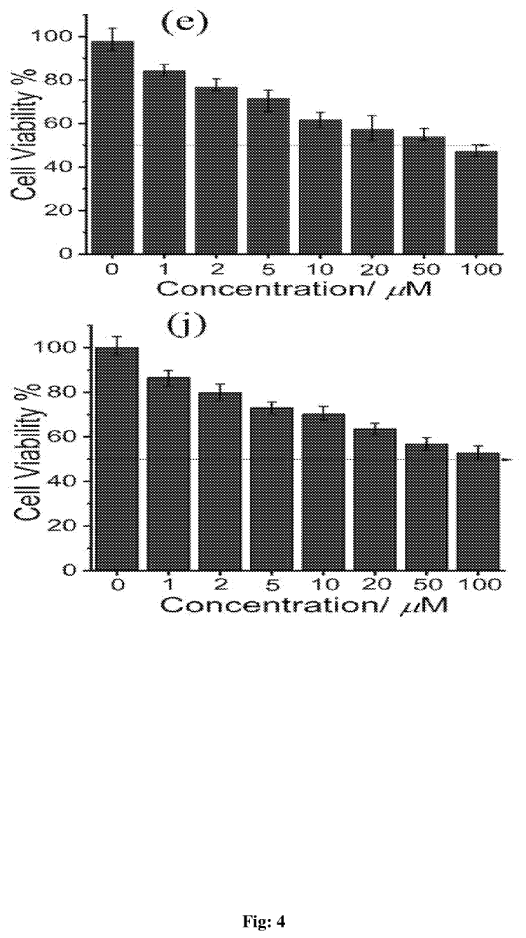

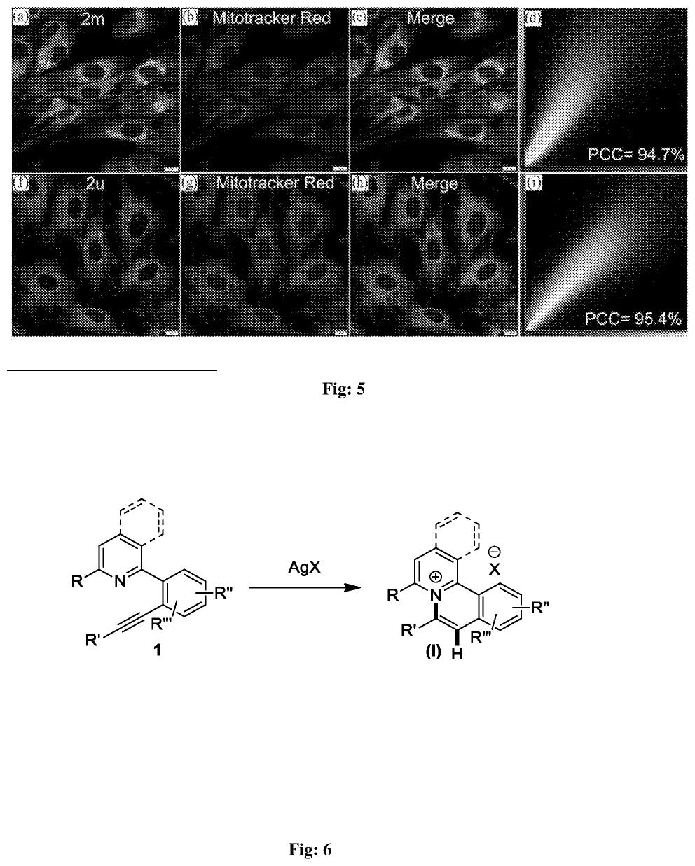

Around 5,000 cells per well were seeded in a 96 well-plate and grown for 24 h in 5% CO₂ at 37° C. in the incubator. After that, dye of interest (2m and 2u) was added from a DMSO stock. The added amount of DMSO was not more than 2 μL. After 24 h of incubation, 20 μL of MTT (3-(4,5-Dimethylthiazol-2-yl)-2,5-Diphenyltetrazolium Bromide) dye solution (from a stock of 5 mg/mL in PBS buffer) was added to each well and incubated for 4 h. The media was removed gently from each well and 100 μL DMSO was added to each well to dissolve the purple color crystal. The absorption at 570 nm was recorded in a Synergy H1 Hybrid Multi-mode microplate reader from Biotek. FIG. 4 depicts the results of cytotoxicity assay of 2m and 2u in different concentrations.

### Confocal Microscopy

All the live-cell imaging experiments were performed with an Olympus FV3000 Confocal Laser Scanning Microscope (LSM) with live-cell imaging set up. The image processing was done with the help of cellSens Dimension software v3.1 (Olympus). For fluorescence imaging, 488 nm (2m and 2u) and 561 nm (MitoTracker Red, ER-Tracker Red, and LysoTracker Red) excitation lasers were used. For 488 nm and 561 nm excitation, the emission windows were kept at 500-540 nm, and 570-670 nm respectively. The confocal aperture was kept at 1.0 Airy Disk (AU) while the dwell time is 8 μs/pixel. The laser power, gain and offset were kept the same for all image acquisitions. The colocalization microscopic experiments have been performed with 2 μM 2m/2u and 0.3 μM commercial tracker dye incubating for 15 minutes at 37° C. in the CO₂ incubator.

The images were acquired in sequential scan mode ensuring no crosstalk between imaging channels. The Pearson's correlation coefficient of 2m and 2u with MitoTracker red was found to be 0.94 and 0.95, respectively. These results illustrate the sensitivity of these lipophilic cationic probes towards the membrane potential change rendering them to be potential candidates for monitoring MMP quantitatively in non-homeostatic and apoptotic conditions.

FIG. 5 represents CLSM images of BHK-21 cells stained (incubation time: 15 min) with (a) 2 μM 2m, (b) 0.3 μM of Mitotracker Red, (c) merge image of (a) and (b), (d) scatter plot showing Pearson's correlation coefficient of 0.94±0.04, (f) 0.2 μM 2u, (g) 0.3 μM of Mitotracker Red, (h) merge image of (f) and (g), (i) scatter plot showing Pearson's correlation coefficient of 0.95±0.02. (Scale bar: 10 μm). The tested compounds displayed rapid and selective mitochondrial localization due to the proton motive force across the mitochondrial membrane.

### ADVANTAGES:

Novel class of N-doped ionic solid state emitters of general formula (I).

Simple and convenient process for the synthesis of library of compounds of general formula (I) employing alkynes.

The photophysical properties can be tuned not only by varying the substituents on the core scaffold but also with a variation of the orientation of the counter anions and the corresponding non-covalent contacts present between the contact ion-pairs.

The compounds of general formula (I) fluoresce in solid state.

1-9. (canceled)

10. A redox-active N-doped ionic solid state luminogen having a pyridinium scaffold that is a compound according to formula (I): 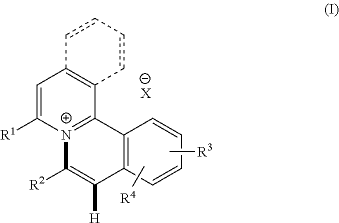
where:
R¹ and R² each independently represents hydrogen, (un)substituted C₁-C₆ alkyl, (un)substituted aryl, (un)substituted alkylaryl (un)substituted heteroaryl, (un)substituted C₃-C₈ saturated or unsaturated cycloalkyls or fused cycloalkyls optionally having heteroatoms selected from the group consisting of O, S, and N;
R³ and R⁴ represent hydrogen, (un)substituted C₁-C₆ alkyl, (un)substituted aryl, (un)substituted alkylaryl, (un)substituted heteroaryl, halogen, nitrile, acetyl, hydroxyl, alkoxy, amino, nitro, carbonyl, ester, thio, or (un)substituted C₃-C₈ saturated or unsaturated cycloalkyls or fused cycloalkyls optionally having heteroatoms selected from the group consisting of O, S, and N;
the dashed ring indicates that a saturated or unsaturated six-membered cyclic ring that may be present; and
X⁻ represents a counterion selected from the group consisting of ⁻OTf, NTF₂⁻, SbF₆⁻, BF₄⁻, or NO₃⁻.

11. The redox-active N-doped ionic solid state luminogen of claim 10, wherein the compound according to formula (I) is compound of formula (Ia): 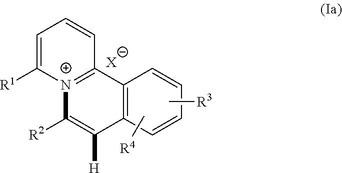
where R¹, R², R³, R⁴, and X are as defined in formula (I).

12. The redox-active N-doped ionic solid state luminogen of claim 10, wherein R¹ is (un)substituted C₁-C₆ alkyl, (un)substituted aryl, or (un)substituted heteroaryl.

13. The redox-active N-doped ionic solid state luminogen of claim 10, wherein R² is (un)substituted C₁-C₆ alkyl, (un)substituted aryl, or (un)substituted cycloalkyl.

14. The redox-active N-doped ionic solid state luminogen of claim 10, wherein R³ is hydrogen, (un)substituted C₁-C₆ alkoxy, (un)substituted C₁-C₆ alkyl, halogen, or (un)substituted aryl.

15. The redox-active N-doped ionic solid state luminogen of claim 10, wherein R⁴ is hydrogen, (un)substituted C₁-C₆ alkoxy, (un)substituted C₁-C₆ alkyl, halogen, or (un)substituted aryl.

16. The redox-active N-doped ionic solid state luminogen of claim 10, wherein the compound is selected from the group consisting of:
i. 4,6-diphenylpyrido[2,1-a]isoquinolin-5-ium trifluoromethansulfonate;
ii. 6-phenyl-4-(p-tolyl)pyrido[2,1-a]isoquinolin-5-iumtrifluoromethane sulfonate;
iii. 4-([1, 1′-biphenyl]-4-yl)-6-phenylpyrido[2,1-a]isoquinolin-5-ium trifluoro methane sulfonate;
iv. 4-(4-cyanophenyl)-6-phenylpyrido[2,1-a]isoquinolin-5-ium trifluoro methane sulfonate;
V. 4-(4-acetylphenyl)-6-phenylpyrido[2,1-a]isoquinolin-5-ium trifluoro methane sulfonate;
vi. 4-(4-methoxyphenyl)-6-phenylpyrido[2,1-a]isoquinolin-5-ium trifluoro methanesulfonate;
vii 4-(4-(9H-carbazol-9-yl)phenyl)-6-phenylpyrido[2, 1-a]isoquinolin-5-ium trifluoro methanesulfonate;
viii. 4-(3,5-dimethoxyphenyl)-6-phenylpyrido[2,1-a]isoquinolin-5-ium trifluoromethanesulfonate;
ix. 4-(anthracen-9-yl)-6-phenylpyrido[2,1-a]isoquinolin-5-ium trifluoro methanesulfonate;
X. 4-(phenanthren-9-yl)-6-phenylpyrido[2,1-a]isoquinolin-5-ium trifluoro methanesulfonate;
xi. 4-(benzo[b]thiophen-2-yl)-6-phenylpyrido[2,1-a]isoquinolin-5-ium trifluoro methanesulfonate;
xii. 4-(9H-carbazol-9-yl)-6-phenylpyrido[2,1-a]isoquinolin-5-ium trifluoro methanesulfonate;
xiii. 4-(4-methoxyphenyl)-10-methyl-6-phenylpyrido[2,1-a]isoquinolin-5-ium trifluoromethanesulfonate;
xiv. 10-(tert-butyl)-4-(4-methoxyphenyl)-6-phenylpyrido[2,1-a]isoquinolin-5-ium trifluoromethanesulfonate;
XV. 4-(4-methoxyphenyl)-6, 10-diphenylpyrido[2, 1-a]isoquinolin-5-ium trifluoromethanesulfonate;
xvi. 4-(4-methoxyphenyl)-10-(naphthalen-1-yl)-6-phenylpyrido[2,1-a]isoquinolin-5-ium trifluoromethanesulfonate;
xvii. 10-chloro-4-(4-methoxyphenyl)-6-phenylpyrido[2,1-a]isoquinolin-5-ium trifluoromethanesulfonate;
xviii. 4-(4-methoxyphenyl)-9-methyl-6-phenylpyrido[2,1-a]isoquinolin-5-ium trifluoromethanesulfonate;
Xix. 9-chloro-4-(4-methoxyphenyl)-6-phenylpyrido[2,1-a]isoquinolin-5-ium trifluoromethanesulfonate;
XX. 4-(4-methoxyphenyl)-8, 10-dimethyl-6-phenylpyrido[2,1-a]isoquinolin-5-ium trifluoromethanesulfonate;
xxi. 8, 10-dimethoxy-4-(4-methoxyphenyl)-6-phenylpyrido[2,1-a]isoquinolin-5- ium trifluoromethanesulfonate;
xxii. 4-(4-methoxyphenyl)-6-(p-tolyl)pyrido[2,1-a]isoquinolin-5-ium trifluoromethanesulfonate;
xxiii. 6-(2-methoxyphenyl)-4-(4-methoxyphenyl)pyrido[2,1-a]isoquinolin-5-ium trifluoromethanesulfonate;
xxiv. 6-(4-chlorophenyl)-4-(4-methoxyphenyl)pyrido[2,1-a]isoquinolin-5-ium trifluoromethanesulfonate;
XXV. 6-(3,5-dimethoxyphenyl)-4-(4-methoxyphenyl)pyrido[2,1-a]isoquinolin-5-ium trifluoromethanesulfonate;
xxvi. 6-(2-chloro-5-methoxyphenyl)-4-(4-methoxyphenyl)pyrido[2,1-a]isoquinolin-5-ium trifluoromethanesulfonate;
xxvii. 4-(4-methoxyphenyl)-6-(naphthalen-1-yl)pyrido[2,1-a]isoquinolin-5-ium trifluoromethanesulfonate;
xxviii. 8-(4-methoxyphenyl)-6-phenylisoquinolino[3,2-a]isoquinolin-7-ium trifluoromethanesulfonate;
XXiX. 4-methyl-6-phenylpyrido[2,1-a]isoquinolin-5-ium;
XXX. 6-cyclopropyl-4-(4-methoxyphenyl)pyrido[2,1-a]isoquinolin-5-ium trifluoromethanesulfonate;
xxxi. 4-(4-methoxyphenyl)-6-phenylpyrido[2,1-a]isoquinolin-5-ium bis((trifluoromethyl)sulfonyl)amide;
xxxii. 4-(4-methoxyphenyl)-6-phenylpyrido[2,1-a]isoquinolin-5-ium hexafluorostibate;
xxxiii. 4-(4-methoxyphenyl)-6-phenylpyrido[2,1-a]isoquinolin-5-ium tetrafluoroborate; and
xxxiv. 4-(4-methoxyphenyl)-6-phenylpyrido[2,1-a]isoquinolin-5-ium nitrate.

17. A process for preparing the redox-active N-doped ionic solid state luminogen according to claim 1, the process comprising:
(a) preparing a 2-(2-alkynylphenyl)pyridine of Formula (I): 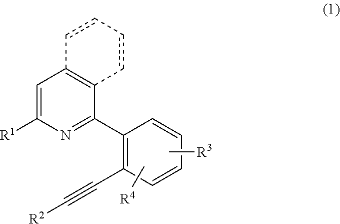
where R¹, R², R³, R⁴, and X are as defined in formula (I) and
(b) promoting an AgX endo-selective hydroamination of the 2-(2-alkynylphenyl)pyridine in a solvent to obtain the compound according to formula (I), wherein AgX is a salt salt selected from the group consisting of AgOTf, AgNTf₂, AgSbF₆, AgBF₄, and AgNO₃.

18. The process of claim 17, wherein (b) comprises heating the 2-(2-alkynylphenyl)pyridine with 1 equivalent of AgX in the solvent at 80° C. for 12 hours.
